(19) United States
(12) Patent Application Publication
(10) Pub. No.: US 2017/0327509 A1
(43) Pub. Date: Nov. 16, 2017

(54) TWO-PHOTON ABSORBING FLUOROPHORES AND METHOD FOR CELLULAR IMAGING USING THE SAME

(71) Applicant: POSTECH ACADEMY-INDUSTRY FOUNDATION, Pohang (KR)

(72) Inventors: Kyo Han AHN, Pohang (KR); Hyunsoo MOON, Uijeongbu (KR); Dokyoung KIM, Pohang (KR); Subhankar SINGHA, Pohang (KR); Basab ROY, Pohang (KR); Sunderraman SAMBASIVAN, Pohang (KR)

(73) Assignee: POSTECH ACADEMY-INDUSTRY FOUNDATION, Pohang (KR)

(21) Appl. No.: 15/512,624

(22) PCT Filed: May 6, 2015
(86) PCT No.: PCT/KR2015/004508

§ 371 Date: Mar. 20, 2017

(30) Foreign Application Priority Data
Sep. 24, 2014 (KR) 10-2014-0127353
Apr. 30, 2015 (KR) 10-2015-0061828

(51) Int. Cl. C07D 487/08; C07D 221/14; C07D 207/08; C07C 231/08; C07C 221/00; A61K 49/00; C07D 295/116; C07C 303/38; C07C 2601/14

(52) U.S. Cl. CPC C07D 487/08; C07D 295/116; C07D 221/14; C07D 207/08; C07C 303/38; C07C 231/08; C07C 221/00; A61K 49/0021; C07C 2601/14

(57) ABSTRACT

The present invention relates to new one-photon or two-photon absorbing fluorophores, a method for preparing the same, and a method for cellular imaging using the same, and more particularly, to new two-photon absorbing fluorophores having higher fluorescence quantum yield and two-photon absorption cross-section value than those of the conventional two-photon absorbing fluorophore, acedan, and thus are promisingly applicable in bioimaging. The design strategy and the compounds according to the present invention may practically utilized for developing new D-π-A fluorophores.

### CROSS-REFERENCE TO RELATED APPLICATION

This application claims priority to and the benefit of Korean Patent Application No. 10-2014-0127353, filed on Sep. 24, 2014, Korean Patent Application No. 10-2015-0061828, filed on Apr. 30, 2015 and International Patent Application No. PCT/KR2015/004508, filed on May 6, 2015, the disclosure of which is incorporated herein by reference in its entirety.

The present invention was undertaken with the support of Korea Health Technology R&D Project No. HI13C1378 grant funded by the Ministry of Health & Welfare of Korea, Global Research Program No. 2014K1A1A2064569 grant funded by the National Research Foundation (NRF) of Korea.

### TECHNICAL FIELD

The present invention relates to new one-photon or two-photon absorbing fluorophores, a method for preparing the same, and a method for cellular imaging using the same.

### BACKGROUND ART

Two-photon microscopy (TPM) is an imaging technique of capturing fluorescence through excitation of fluorophores by using two photons having energy equal to half of the energy of a photon used in one-photon microscopy (OPM).

TPM allows excitation of fluorophores by light with energy equal to half of that used in OPM (i.e., with a wavelength that is two times longer), and thus offers the advantages of deeper tissue penetration, and less photodamage and photobleaching of living tissues and cells in bioimaging. Also, TPM is less influenced by autofluorescence generated from intrinsic biomoleucles and is able to provide very high resolution images since excitation occurs only at the focal point (Zipfel, W. R. et al. Nat. Biotechnol. 2003, 21, 1369; Helmchen, F. et al. Nat. Methods, 2005, 2, 932; Willams, W. R. et al. Curr. Opin. Chem. Biol. 2001, 5, 603).

Therefore, efficient two-photon absorbing fluorophores used together with two-photon microscopy to obtain images in vivo are also very important materials in the bioimaging field. An efficient two-photon absorbing fluorophore needs to have a large two-photon absorption cross-section value within a proper biological optical window wavelength region (800 to 1000 nm) to minimize auto fluorescence of living tissue and also needs to ensure photostability, permeability into biological matters, and biocompatibility.

The two-photon absorbing fluorophores satisfying such requirements for bioimaging are limited in number, and generally, D-π-A dipolar dyes that have an electron donor (D) and an electron acceptor (A) in an aromatic ring (π-system) have been widely used. —As a representative example of the dipolar dyes, 1-(6-dimethylaminonaphthalen-2-yl)ethanone (acedan) represented by Formula 19 is used to obtain bright images by two-photon microscopy in living cells and tissues due to high photostability and a quite large two-photon absorption cross-section value (Kim, H. M. et al. Angew. Chem. Int. Ed. 2007. 46, 3460; Kim, H. M. et al. Angew. Chem. Int. Ed. 2008, 47, 5167; Kim, H. M. et al. Angew. Chem. Int. Ed. 2007, 46, 7445).

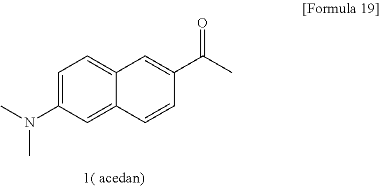 [SMILES: CC(=O)c1ccc2cc(N(C)C)ccc2c1]

However, such D-π-A dipolar dyes generate intramolecular charge transfer excited state, thus resulting the fluorescence property highly sensitive towards the environment polarity (polarity of a solvent), and such a property is significantly applied in the detection of a substrate accompanying polarity changes in vivo.

On the other hand, these dipolar dyes have a critical disadvantage of poor fluorescence intensities in aqueous solution, resulting in low fluorescence quantum yield and two-photon absorption cross-section value (MacGregor, R. B. et al. Nature 1986, 319, 70; Hutterer, R. et al. J. Fluoresc. 1998, 8, 365; Gaus, K. et al. Proc. Natl. Acad. Sci. USA 2003, 100, 15554).

That is, such dipolar dyes need to overcome the environmental polarity sensitivity which causes poor fluorescence intensities in aqueous solution.

### DISCLOSURE

### Technical Problem

Therefore, to overcome the above-mentioned problems of the conventional art, the inventors developed new two-photon absorbing fluorophores having high fluorescence quantum yield and a two-photon absorption cross-section value, thereby completing the present invention.

Accordingly, an objective of the present invention is directed to providing compounds represented by Formula 1 or a pharmaceutically acceptable salt thereof.

Another objective of the present invention is directed to providing a method for cellular imaging using the compounds.

Still another objective of the present invention is directed to providing a method for preparing the compound.

However, technical problems to be solved in the present invention are not limited to the above-described problems, and other problems which are not described herein will be fully understood by those of ordinary skill in the art from the following description.

### Technical Solution

To achieve the above-mentioned objectives of the present invention, the present invention provides compounds represented by Formula 1.

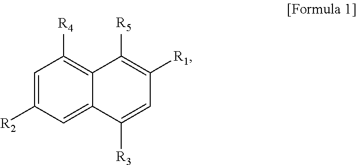

wherein R₁ is hydrogen or

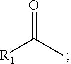

R₂ is hydrogen,

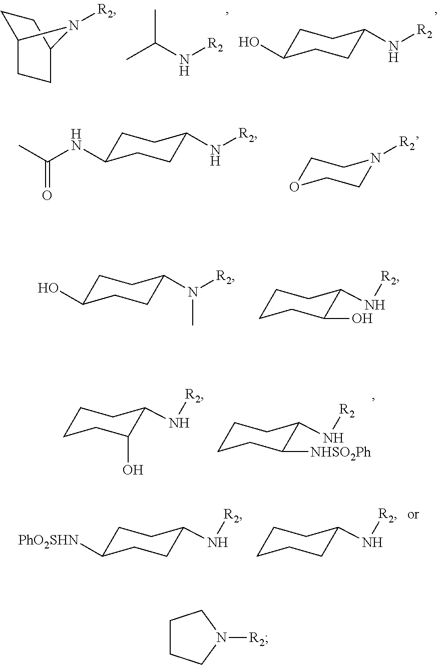

R₃ is hydrogen or

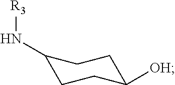

and

R₄ and R₅ are hydrogen or

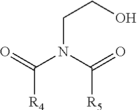

linked by a 6-membered ring.

In one embodiment of the present invention, the compound may be one-photon absorbing fluorophores or two-photon absorbing fluorophores.

In addition, the present invention provides a method for cellular imaging using the compounds or a pharmaceutically acceptable salt thereof.

In one embodiment of the present invention, the method may include treating cells with the compounds or a pharmaceutically acceptable salt thereof and measuring fluorescence using a fluorescence microscope.

In another embodiment of the present invention, the fluorescence microscope may be a one-photon fluorescence microscope or a two-photon fluorescence microscope.

Further, the present invention provides a method for preparing a compound of Formula 2, which includes the following steps: 1) synthesizing 4-(6-bromonaphthalene-2-ylamino)cyclohexanol by adding trans-4-aminocyclohexanol and sodium metabisulfite to 6-bromo-2-naphthol; 2) synthesizing 4-(6-bromonaphthalene-2-ylamino)cyclohexyl methanesulfonate by adding triethylamine and methanesulfonylchloride to the 4-(6-bromonaphthalene-2-ylamino)cyclohexanol; 3) synthesizing 7-(6-bromonaphthalene-2-yl)7-azacyclo[2.2.1]heptane by adding dimethylformamide to the 4-(6-bromonaphthalene-2-ylamino)cyclohexyl methanesulfonate; and 4) adding palladium(II)acetate, diphenylphosphinopropane, ethyleneglycolvinylether, and triethylamine to the 7-(6-bromonaphthalene-2-yl)7-azacyclo[2.2.1]heptane.

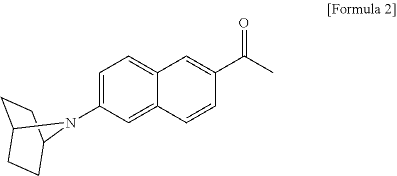 [SMILES: CC(=O)c1ccc2cc(N3C4CCC3CC4)ccc2c1]

In addition, the present invention provides a method for preparing a compound of Formula 3, which includes the following steps: 1) synthesizing 6-bromo-N-isopropylnaphthalene-2-amine by adding isopropylamine and sodium metabisulfite to 6-bromo-2-naphthol; and 2) adding palladium(II)acetate, diphenylphosphinopropane, ethyleneglycolvinylether, and triethylamine to the 6-bromo-N-isopropylnaphthalene-2-amine

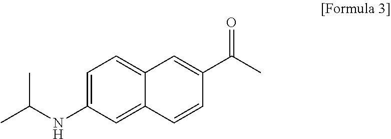 [SMILES: CC(=O)c1ccc2cc(NC(C)C)ccc2c1]

In addition, the present invention provides a method for preparing a compound of Formula 5, which includes the following steps: 1) synthesizing 4-(6-bromonaphthalene-2-ylamino)cyclohexanol by adding trans-4-aminocyclohexanol and sodium metabisulfite to 6-bromo-2-naphthol; and 2) adding palladium(II)acetate, diphenylphosphinopropane, ethyleneglycolvinylether, and triethylamine to the 4-(6-bromonaphthalene-2-ylamino)cyclohexanol.

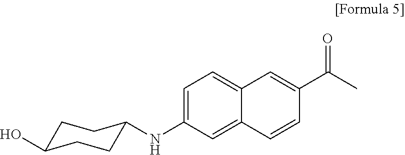 [SMILES: CC(=O)c1ccc2cc(NC3CCC(O)CC3)ccc2c1]

In addition, the present invention provides a method for preparing a compound of Formula 9, which includes adding a formaldehyde aqueous solution, sodium cyanoborohydride, and zinc chloride to the compound of Formula 5.

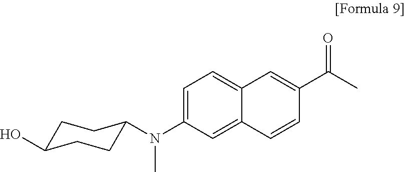 [SMILES: CC(=O)c1ccc2cc(N(C)C3CCC(O)CC3)ccc2c1]

In addition, the present invention provides a method for preparing a compound of Formula 6, which includes the following steps: 1) synthesizing 1-(6-hydroxynaphthalen-2-yl)ethanone by adding palladium(II)acetate, diphenylphosphinopropane, ethyleneglycolvinylether, and triethylamine to 6-bromo-2-naphthol; 2) synthesizing 1-(6-(4-aminocyclohexylamino)naphthalene-2-yl)ethanone by adding trans-1,4-diaminocyclohexane and sodium metabisulfite to the 1-(6-hydroxynaphthalen-2-yl)ethanone; and 3) adding acetic anhydride to the 1-(6-(4-aminocyclohexylamino)naphthalene-2-yl)ethanone.

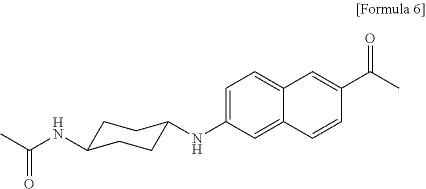 [SMILES: CC(=O)NC1CCC(Nc2ccc3cc(C(C)=O)ccc3c2)CC1]

In addition, the present invention provides a method for preparing a compound of Formula 8, which includes the following steps: 1) synthesizing 4-(6-bromonaphthalene-2-yl)morpholine by adding morpholine and sodium metabisulfite to 6-bromo-2-naphthol; and 2) adding palladium(II)acetate, diphenylphosphinopropane, ethyleneglycolvinylether, and triethylamine to the 4-(6-bromonaphthalene-2-yl)morpholine.

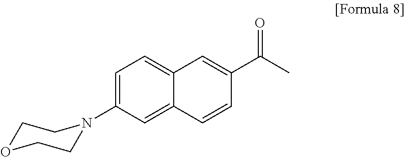 [SMILES: CC(=O)c1ccc2cc(N3CCOCC3)ccc2c1]

In addition, the present invention provides a method for preparing a compound of Formula 10, which includes adding trans-4-aminocyclohexanol to 6-bromo-2-(2-hydroxyethyl)-1H-benzo[de]isoquinoline-1,3 (2H)-dione.

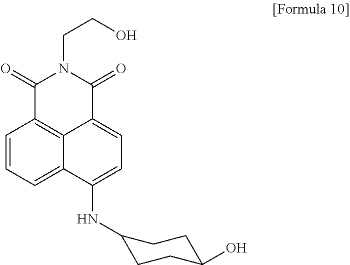 [SMILES: O=C1c2cccc3c(NC4CCC(O)CC4)ccc(c23)C(=O)N1CCO]

In addition, the present invention provides a method for preparing a compound of Formula 13, which includes adding sodium metabisulfite and (1S,2S)-2-aminocyclohexanol to 1-(6-hydroxynaphthalen-2-yl)ethanone.

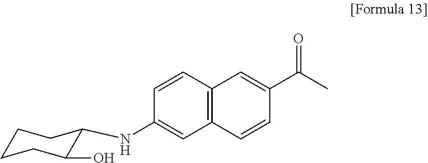 [SMILES: CC(=O)c1ccc2cc(NC3CCCCC3O)ccc2c1]

In addition, the present invention provides a method for providing a compound of Formula 14, which includes adding sodium metabisulfite and (1R,2S)-2-aminocyclohexanol to 1-(6-hydroxynaphthalen-2-yl)ethanone.

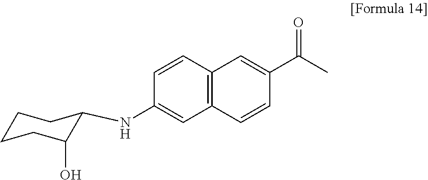 [SMILES: CC(=O)c1ccc2cc(NC3CCCCC3O)ccc2c1]

In addition, the present invention provides a method for preparing a compound of Formula 15, which includes the following steps: 1) synthesizing 1-(6-(((1S,2S)-2-aminocyclohexyl)amino)naphthalene-2-yl)ethanone by adding sodium metabisulfite and (1R,2R)-cyclohexane-1,2-diamine to 1-(6-hydroxynaphthalen-2-yl)ethanone; and 2) adding benzenesulfonyl chloride and triethylamine to the 1-(6-(((1S,2S)-2-aminocyclohexyl)amino)naphthalene-2-yl)ethanone.

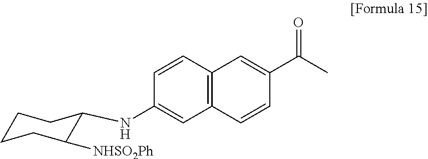

In addition, the present invention provides a method for preparing a compound of Formula 16, which includes the steps: 1) synthesizing 1-(6-(((1R,4R)-4-aminocyclohexyl)amino)naphthalene-2-yl)ethanone by adding sodium metabisulfite and (1R,4R)-cyclohexane-1,4-diamine to 1-(6-hydroxynaphthalen-2-yl)ethanone; and 2) adding benzenesulfonyl chloride and triethylamine to the 1-(6-(((1R,4R)-4-aminocyclohexyl)amino)naphthalene-2-yl)ethanone.

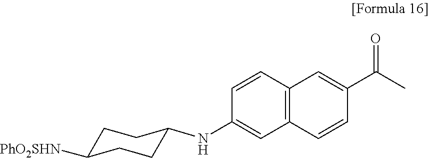

In addition, the present invention provides a method for preparing a compound of Formula 17, which includes adding sodium metabisulfite and cyclohexaneamine to 1-(6-hydroxynaphthalen-2-yl)ethanone.

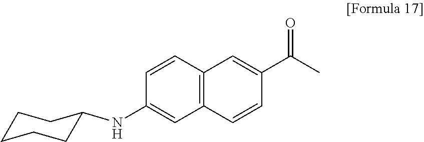 [SMILES: CC(=O)c1ccc2cc(NC3CCCCC3)ccc2c1]

In addition, the present invention provides a method for preparing a compound of Formula 18, which includes adding sodium metabisulfite and pyrrolidine to 1-(6-hydroxynaphthalen-2-yl)ethanone.

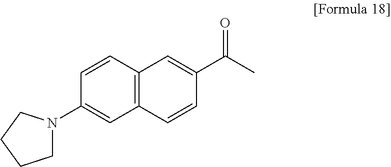 [SMILES: CC(=O)c1ccc2cc(N3CCCC3)ccc2c1]

### Advantageous Effects

Substituents included in a the newly developed compounds of the present invention are expected to be useful for the development of new bright D-π-A fluorophores, and particularly, the introduction of a 4-hydroxycyclohexylamino group as shown in a compound of Formula 5 to different D-π-A fluorophores, is expected to resulting the development of fluorophores having higher fluorescence quantum yield and two-photon absorption cross-section value in aqueous solution.

### DESCRIPTION OF DRAWINGS

FIG. 1 shows the structural formulas of Compound 1 (upper left panel) and Compound 5 (lower left panel), and two-photon fluorescence microscopic images (right) of NIH3T3 cells treated with Compounds 1 and 5.

FIG. 2 shows absorbance spectra for Compounds 1 to 9 at the concentration of 10 μM in 4-(2-hydroxyethyl)-1-piperazineethanesulfonic acid (HEPES) buffer (pH 7.4, containing 1% dimethyl sulfoxide (DMSO), left) and water (containing 1% DMSO, right), respectively.

FIG. 3 shows absorbance spectra for Compounds 1 to 9 at the concentration of 10 μM in ethanol (EtOH) (left) and acetonitrile (CH₃CN) (right), respectively.

FIG. 4 shows absorbance spectra for Compounds 1 to 9 at the concentration of 10 μM in N,N-dimethylformamide (DMF) (left) and dichloromethane (CH₂Cl₂) (right), respectively.

FIG. 5 shows absorbance spectra for Compounds 1 to 9 at the concentration of 10 μM in cyclohexane (c-C₆H₁₂).

FIG. 6 shows maximum absorbance wavelengths of Compounds 1 to 9 at the concentration of 10 μM in HEPES buffer (containing 1% DMSO, pH 7.4), water (containing 1% DMSO), ethanol, acetonitrile, dimethylformamide, dichloromethane, and cyclohexane, respectively.

FIG. 7 shows molar extinction coefficients of Compounds 1 to 9 at the concentration of 10 μM in HEPES buffer (containing 1% DMSO, pH 7.4), water (containing 1% DMSO), ethanol, acetonitrile, dimethylformamide, dichloromethane, and cyclohexane, respectively.

FIG. 8 shows absorbance spectra for Compounds 1 to 9 at the concentration of 10 μM in HEPES buffer (containing 1% DMSO, pH 7.4), water (containing 1% DMSO), ethanol, acetonitrile, dimethylformamide, dichloromethane, and cyclohexane, respectively.

FIG. 9 shows fluorescence spectra for Compounds 1 to 9 at the concentration of 10 μM in 4-(2-hydroxyethyl)-1-piperazineethanesulfonic acid (HEPES) buffer (pH 7.4, containing 1% dimethyl sulfoxide (DMSO), left) and water (containing 1% DMSO, right), respectively.

FIG. 10 shows fluorescence spectra for Compounds 1 to 9 at the concentration of 10 μM in ethanol (EtOH) (left) and acetonitrile (CH₃CN) (right), respectively.

FIG. 11 shows fluorescence spectra for Compounds 1 to 9 at the concentration of 10 μM in N,N-dimethylformamide (DMF) (left) and dichloromethane (CH₂Cl₂) (right), respectively.

FIG. 12 shows fluorescence spectra for Compounds 1 to 9 at the concentration of 10 μM in cyclohexane.

FIG. 13 shows a comparison of fluorescence intensities between Compounds 1 to 9 at the concentration of 10 μM in HEPES buffer (containing 1% DMSO, pH 7.4) and water (containing 1% DMSO) (a), a fluorescence image of Compound 1 at the concentration of 10 μM in water (b, left), and a fluorescence image of Compound 5 at the concentration of 10 μM in water (b, right).

FIG. 14 shows maximum emission wavelengths of Compounds 1 to 9 at the concentration of 10 μM in HEPES buffer (containing 1% DMSO, pH 7.4), water (containing 1% DMSO), ethanol, acetonitrile, dimethylformamide, dichloromethane, and cyclohexane, respectively.

FIG. 15 shows fluorescence quantum yield of Compounds 1 to 9 in dichloromethane, acetonitrile, and aqueous (containing 1% DMSO).

FIG. 16 shows fluorescence spectra obtained from one-photon (black) and two-photon (red) excitation of Compound 1 in water (containing 1% DMSO).

FIG. 17 shows fluorescence spectra obtained from one-photon (black) and two-photon (red) excitation of Compound 1 in acetonitrile.

FIG. 18 shows fluorescence spectra obtained from one-photon (black) and two-photon (red) excitation of Compound 1 in dichloromethane.

FIG. 19 shows fluorescence spectra obtained from one-photon (black) and two-photon (red) excitation of Compound 5 in water (containing 1% DMSO).

FIG. 20 shows fluorescence spectra obtained from one-photon (black) and two-photon (red) excitation of Compound 5 in acetonitrile.

FIG. 21 shows fluorescence spectra obtained from one-photon (black) and two-photon (red) excitation of Compound 5 in dichloromethane.

FIG. 22 shows fluorescence spectra obtained from one-photon (black) and two-photon (red) excitation of Compound 6 in water (containing 1% DMSO).

FIG. 23 shows fluorescence spectra obtained from one-photon (black) and two-photon (red) excitation of Compound 6 in acetonitrile.

FIG. 24 shows fluorescence spectra obtained from one-photon (black) and two-photon (red) excitation of Compound 6 in dichloromethane.

FIG. 25 shows fluorescence spectra obtained from one-photon (black) and two-photon (red) excitation of Compound 7 in water (containing 1% DMSO).

FIG. 26 shows fluorescence spectra obtained from one-photon (black) and two-photon (red) excitation of Compound 7 in acetonitrile.

FIG. 27 shows fluorescence spectra obtained from one-photon (black) and two-photon (red) excitation of Compound 7 in dichloromethane.

FIG. 28 shows two-photon absorption cross-section values obtained when each of Compounds 1, 5, 6, and 7 is excited in dichloromethane, acetonitrile, and water (containing 1% DMSO) at 740 nm, 760 nm, and 780 nm.

FIG. 29 shows two-photon absorption cross-section values obtained when each of Compounds 1, 5, 6, and 7 is excited in dichloromethane, acetonitrile, and water (containing 1% DMSO) at 740 nm.

FIG. 30 shows two-photon fluorescence microscopic images of NIH3T3 cells treated with Compounds 1 and 5.

FIG. 31 shows absorbance spectra for Compound 10 (red), Compound 11 (blue), and Compound 12 (black) in water (a, containing 1% DMSO), acetonitrile (b), and dichloromethane (c).

FIG. 32 shows fluorescence spectra for Compound 10 (red), Compound 11 (blue) and Compound 12 (black) in water (a, containing 1% DMSO), acetonitrile (b), and dichloromethane (c)

FIG. 33 shows the molar extinction coefficients (ε), the maximum absorption wavelengths (λ_{abs}) and the maximum emission wavelengths (λₑₘ) for Compounds 10, 11, and abs, 12 in water, acetonitrile, and dichloromethane.

FIG. 34 shows fluorescence quantum yields of Compounds 10, 11, and 12 in water, acetonitrile, and dichloromethane.

FIG. 35 shows two-photon absorption cross-section values obtained when each of Compound 10, Compound 11, and Compound 12 is excited in water, acetonitrile, and dichloromethane at 800 nm, 820 nm, and 840 nm.

FIG. 36 shows two-photon fluorescence microscopic images (a) for HeLa cells treated with each of Compounds 1, 5, 10, and 12 and relative fluorescence intensities (b) of the microscopic images.

FIG. 37 shows two-photon fluorescence microscopic images (a) obtained when mouse brain, liver and kidney tissue treated with each of Compounds 1 and 5 were excited at 740 nm and relative fluorescence intensities (b) of the microscopic images.

FIG. 38 shows two-photon fluorescence microscopic images (a) obtained when mouse brain, liver and kidney tissue treated with each of Compounds 1 and 5 were excited at 880 nm and relative fluorescence intensities (b) of the microscopic images.

FIG. 39 shows two-photon fluorescence microscopic images (a) obtained when mouse brain, liver and kidney tissue treated with each of Compounds 10 and 12 were excited at 900 nm and relative fluorescence intensities (b) of the microscopic images.

FIG. 40 shows fluorescence intensities of Compounds 1, 5, 13, 14, 15, 16, 17, and 18 at the concentration of 1 μM in water (containing 1% DMSO).

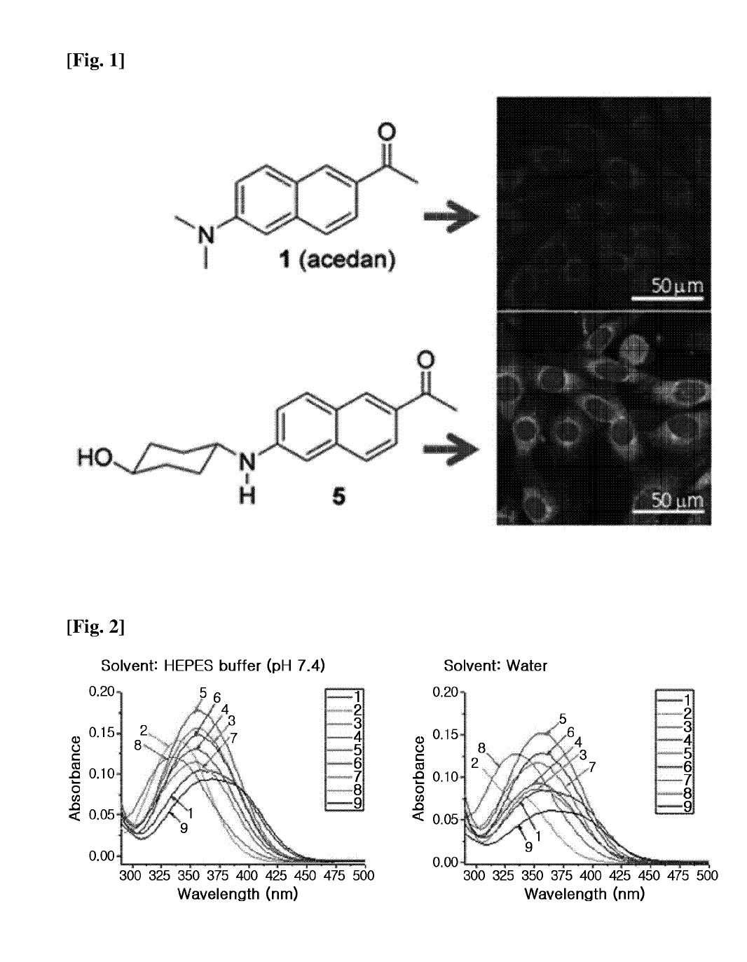

### MODES OF THE INVENTION

The present invention is characterized by providing new one-photon absorbing fluorophores and/or two-photon absorbing fluorophores, which is a compound represented by Formula 1 as shown below.

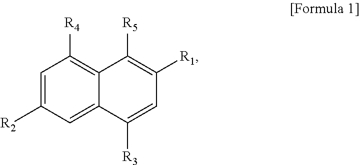

Here, R₁ may be hydrogen or

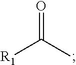

R₂ may be hydrogen,

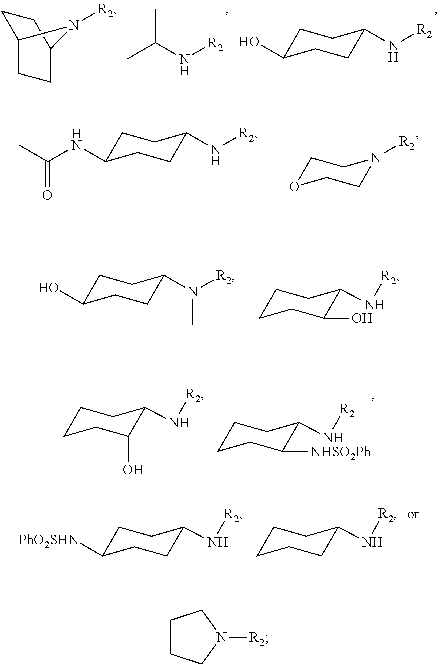

R₃ may be hydrogen or

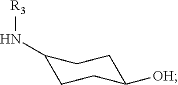

and

R₄ and R₅ may be hydrogen or

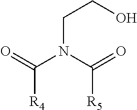

linked by a 6-membered ring, but the present invention is not limited thereto.

Most preferably, Compound 1 may be a substituent represented by Formulas 2, 3, 5, 6, 8, 9, 10, 13, 14, 15, 16, 17, or 18 as shown below.

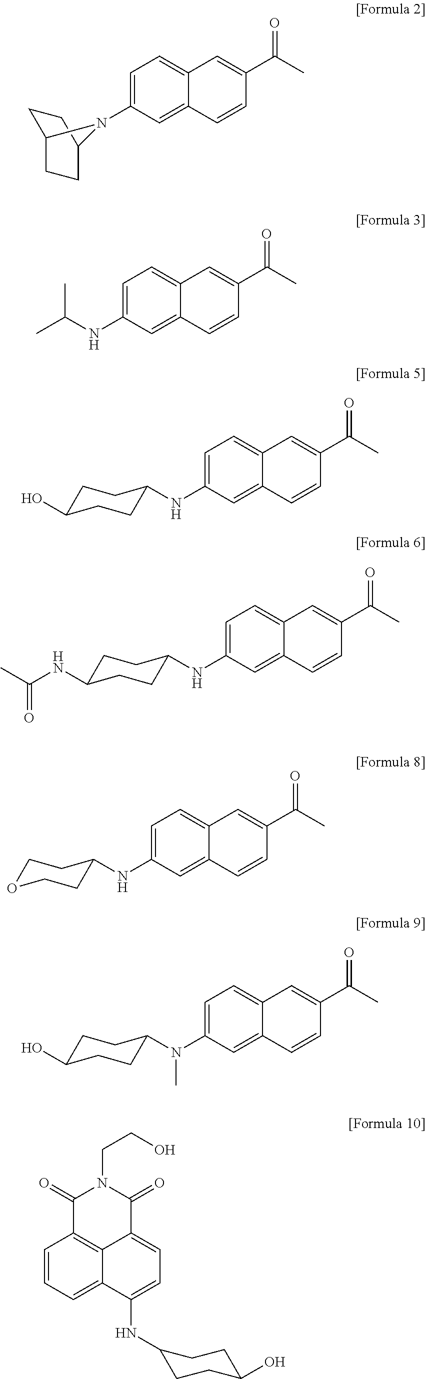

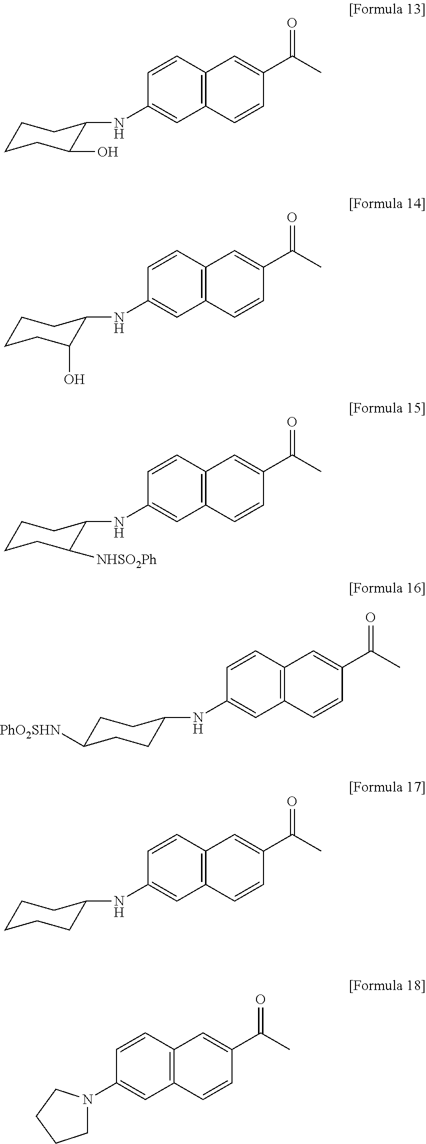

In one embodiment of the present invention, it was confirmed that compounds of the present invention have two-photon absorption cross-section values higher than those of conventional two-photon absorbing fluorophores and thus are able to provide excellent bright fluorescent images through bioimaging under two-photon microscopy (Experimental Examples 1 to 6).

Therefore, the present invention may provide a method for cellular imaging using the compounds of the present invention.

Herein after, exemplary examples will be provided to help in understanding of the present invention. However, the following examples are merely provided to facilitate understanding of the present invention, and the scope of the present invention is not limited to the following examples.

### Example 1

### Synthesis of Compound 2

A general synthetic pathway of Compound 2 is shown in Scheme 1.

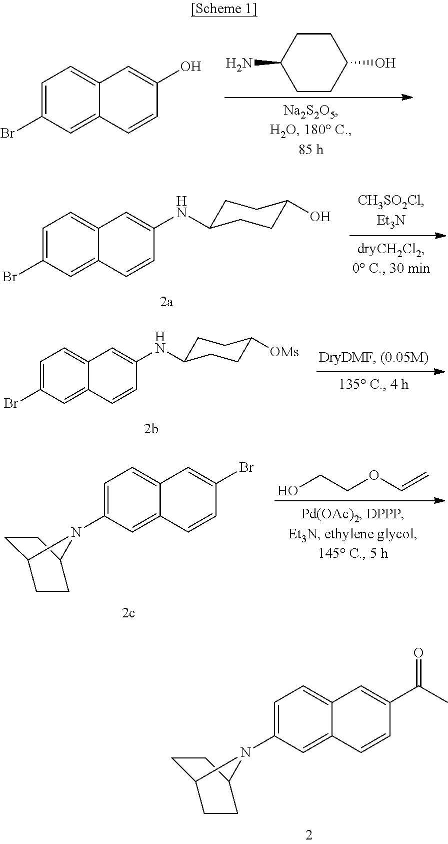

### <1-1> Synthesis of Compound 2a (4-(6-bromonaphthalen-2-ylamino)cyclohexanol)

Compound 2a, 4-(6-bromonaphthalene-2-ylamino)cyclohexanol, was synthesized by the inventors.

Specifically, water (15 mL) was added to a sealed tube containing starting materials for synthesis such as 6-bromo-2-naphthol (1.5 g, 6.72 mmol, Sigma-aldrich B73406), trans-4-aminocyclohexanol (1.55 g, 13.45 mmol), and sodium metabisulfite (2.56 g, 13.45 mmol), and the tube was closed. The resulting mixture was stirred at 180° C. for 96 hours using a silicone oil container. After the mixture was cooled to room temperature (25° C.), the container was opened to dilute the mixture with ethyl acetate (EtOAc, 300 mL). An organic layer was washed with water (80 mL), a 5% sodium bicarbonate aqueous solution (50 mL), and a saturated saline solution (50 mL) and dehydrated with anhydrous sodium sulfate (Na₂SO₄, 30 g). The solvent was removed under a reduced pressure condition of 40 mbar, and the resulting product was purified by column chromatography through a silica gel (Merck-silicagel 60, 230-400 mesh; using 5% EtOAc/hexane as a developer), thereby obtaining a brown solid, Compound 2a (600 mg, 38%; 27% 6-bromo-2-naphthol was recovered).

¹H NMR (CDCl₃, 300 MHz, 298 K, δ): 7.79 (d, J=1.5 Hz, 1H), 7.53-7.38 (m, 1H), 6.84 (dd, J=8.7, 2, 1 Hz, 1H), 6.74 (d, J=2.1 Hz, 1H), 3.76-3.66 (m, 2H), 3.43-3.33 (m, 1H), 2.24-2.19 (m, 2H), 2.08-2.03 (m, 2H), 1.55-1.42 (m, 4H), 1.33-1.19 (m, 4H); ¹³C NMR (CDCl₃, 75 MHz, 298 K, δ): 145.4, 133.9, 129.7, 128.5, 128.3, 127.6, 119.2, 115.1, 104.7, 70.3, 51.3, 34.2, 31.1; IR (KBr, cm⁻¹): 2934, 1625, 1590; HRMS (FAB): m/z calcd for C₁₆H₁₈BrNO [M⁺] 319.0572, [M⁺+2] 321.0553; found 319.0570 [M⁺], 321.0558 [M⁺+2]; calcd for C₁₆H₁₉BrNO [MH⁺] 320.0604, [MH⁺+2] 322.0585; found 320.0607 [MH⁺], 322.0673 [MH⁺+2]; mp: 139-141° C.

### <1-2> Synthesis of Compound 2b (4-(6-bromonaphthalen-2-ylamino)cyclohexyl methanesulfonate)

Compound 2b, 4-(6-bromonaphthalen-2-ylamino)cyclohexyl methanesulfonate, was synthesized by the inventors.

Specifically, Compound 2a (474 mg, 1.48 mmol) obtained in Example 1-1 was dissolved in anhydrous dichloromethane (CH₂Cl₂, 10 mL), and triethylamine (Et₃N, 268 μL, 1.93 mmol) obtained through distillation was added thereto. The resulting mixture was cooled to 0° C. using ice, and a solution prepared by dissolving methanesulfonyl chloride (137 μL, 1.78 mmol) in anhydrous dichloromethane (1 mL) was slowly added dropwise for 5 minutes. The resulting mixture was stirred at 0° C. for 30 minutes (the reaction progress was checked by thin-layer chromatography (TLC)), cold water (10 mL) was added to terminate the reaction, and then extraction was performed with ethyl acetate (2×100 mL). An organic layer was washed with water (50 mL) and a saturated saline solution (50 mL) and dehydrated with anhydrous sodium sulfate (10 g). The solvent was removed under a reduced pressure condition of 40 mbar, and the resulting product was purified by column chromatography through a silica gel (Merck-silicagel 60, 230-400 mesh; using 5% hexane/CH₂Cl₂ as a developer), thereby obtaining a brown solid, Compound 2b (384 mg, 65%).

¹H NMR (CDCl₃, 300 MHz, 298 K, δ): 7.8 (d, J=1.8 Hz, 1H), 7.54-7.39 (m, 3H), 6.85 (dd, J=8.7, 2.1 Hz, 1H), 4.77-4.68 (m, 1H), 3.48-3.39 (m, 1H), 3.04 (s, 3H), 2.3-2.21 (m, 4H), 1.86-1.72 (m, 2H), 1.48-1.3 (m, 2H).

### <1-3> Synthesis of Compound 2c (7-(6-bromonaphthalen-2-yl)-7-azabicyclo[2.2.1]heptane)

Compound 2c, 7-(6-bromonaphthalen-2-yl)-7-azabicyclo[2.2.1]heptane, was synthesized by the inventors.

Specifically, Compound 2b (384 mg, 1.48 mmol) obtained through Example 1-2 and anhydrous dimethylformamide (N,N-dimethylformamide, DMF, 20 mL) were added to a oven-dried flask and charged with argon gas. The resulting mixture was stirred at 135° C. for 4 hours using a silicone oil container (the reaction progress was confirmed by TLC). The mixture was cooled to room temperature and diluted with ethyl acetate (300 mL). An organic layer was washed with water (3×50 mL) and a saturated saline solution (50 mL) and dehydrated with anhydrous sodium sulfate (30 g). The solvent was removed under a reduced pressure condition of 40 mbar, and the resulting product was purified by column chromatography through a silica gel (Merck-silicagel 60, 230-400 mesh; using EtOAc/hexane as a developer), thereby obtaining a yellow solid, Compound 2c (228 mg, 89%).

¹H NMR (CDCl₃, 300 MHz, 298 K, δ): 7.83 (d, J=1.8 Hz, 1H), 7.58 (d, J=9.0 Hz, 1H), 7.5 (d, J=8.7 Hz, 1H), 7.42 (dd, J=8.7, 1.8 Hz, 1H), 7.21 (dd, J=9.0, 2.1 Hz, 1H), 7.07 (d, J=2.1 Hz, 1H), 4.3-4.29 (m, 2H), 1.85-1.82 (m, 4H), 1.49-1.47 (m, 4H); ¹³C NMR (CDCl₃, 75 MHz, 298 K, δ): 146.5, 133.5, 129.6, 129.5, 129.4, 128.2, 120.2, 116.3, 110.7, 58.3, 29; IR (KBr, cm⁻¹): 2945, 1621; HRMS: m/z calcd for C₁₆H₁₆BrN [M⁺] 301.0466, [M⁺+2] 303.0447; found 301.0462 [M⁺], 303.0433 [M⁺+2]; calcd for C₁₆H₁₇BrN [MH⁺]302.0499, [MH⁺+2] 304.0479; found 302.0511 [MH⁺], 304.0515 [MH⁺+2]; mp: 181-183° C.

### <1-4> Synthesis of Compound 2 (1-(6-(7-azabicyclo[2.2.1]heptan-7-yl)naphthalen-2-yl)ethanone)

Compound 2, 1-(6-(7-azabicyclo[2.2.1]heptan-7-yl)naphthalen-2-yl)ethanone, was synthesized by the inventors.

Specifically, Compound 2c obtained in Example 1-3 (184 mg, 0.61 mmol), palladium(II) acetate (Pd(OAc)₂, 6.8 mg, 0.03 mmol), diphenylphosphinopropane (DPPP, 25.2 mg, 0.06 mmol), and ethyleneglycol (1.5 mL) were added to an oven-dried flask with two necks and charged with argon gas. After oxygen present in the mixture was removed by adding the argon gas to the mixture, ethyleneglycol vinyl ether (279 μL, 1.53 mmol) and Et₃N (255 μL, 1.83 mmol) obtained by distillation were sequentially added thereto. The resulting mixture was stirred at 145° C. for 5 hours using a silicone oil container. The mixture was cooled to room temperature, and stirred with a 6N hydrochloric acid (HCl) aqueous solution (4 mL) at 60° C. for 4 hours. The mixture was cooled to room temperature, and diluted with ethyl acetate (100 mL). An organic layer was washed with water (50 mL), a 5% sodium bicarbonate aqueous solution (50 mL), and a saturated saline solution (50 mL) and dehydrated with anhydrous sodium sulfate (10 g). The solvent was removed under a reduced pressure condition of 40 mbar, and the resulting product was purified by column chromatography through a silica gel (Merck-silicagel 60, 230-400 mesh; using EtOAc/hexane as a developer), thereby obtaining a yellow solid, Compound 2 (100 mg, 62%). By further purification using recrystallization (using 3% CH₂Cl₂/hexane as a solvent), a yellow solid, Compound 2 (32 mg, 20%), was obtained.

¹H NMR (CDCl₃, 300 MHz, 298 K, δ): 8.32 (d, J=1.5 Hz, 1H), 7.93 (dd, J=8.7, 1.8 Hz, 1H), 7.78 (d, J=9.0 Hz, 1H), 7.64 (d, J=8.7 Hz, 1H), 7.24 (dd, J=8.7, 2.1 Hz, 1H), 7.11 (d, J=2.4 Hz, 1H), 4.37-4.34 (m, 2H), 2.67 (s, 3H), 1.87-1.84 (m, 4H), 1.54-1.5 (m, 4H); ¹³C NMR (CDCl₃, 75 MHz, 298 K, δ): 198.0, 148.6, 137.7, 131.9, 131.0, 130.4, 127.0, 126.7, 124.7, 119.8, 110.3, 58.3, 29.0, 26.7; IR (KBr, cm⁻¹): 1670; HRMS: m/z calcd for C₁₈H₁₉NO [M⁺] 265.1467, C₁₈H₂₀NO [MH⁺] 266.1545; found 265.1467 [M⁺], 266.1547 [MH⁺]; mp: 118-120° C.

### Example 2

### Synthesis of Compound 3

A general synthetic pathway of Compound 3 is shown in Scheme 2.

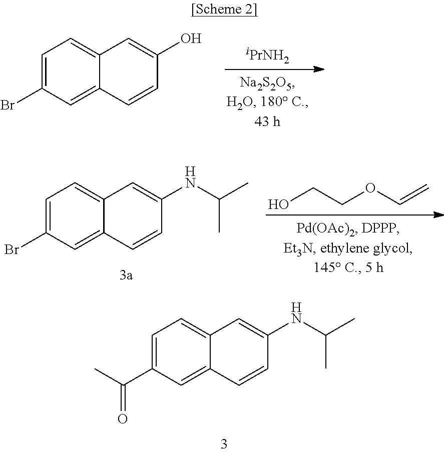

### <2-1> Synthesis of Compound 3a (6-bromo-N-isopropylnaphthalen-2-amine)

Compound 3a, 6-bromo-N-isopropylnaphthalen-2-amine, was synthesized by the inventors.

Specifically, water (10 mL) was added to a sealed tube containing starting materials for synthesis such as 6-bromo-2-naphthol (1.0 g, 4.50 mmol), isopropyl amine (4 mL, 48.86 mmol), and sodium metabisulfite (1.3 g, 6.80 mmol), and the tube was closed. The resulting mixture was stirred at 180° C. for 48 hours using a silicone oil container. The mixture was cooled to room temperature and diluted with ethyl acetate (300 mL). An organic layer was washed with water (80 mL), a 5% sodium bicarbonate aqueous solution (50 mL), and a saturate saline solution (50 mL) and dehydrated with anhydrous sodium sulfate (30 g). The solvent was removed under a reduced pressure condition of 40 mbar, and the resulting product was purified by column chromatography through a silica gel (Merck-silicagel 60, 230-400 mesh; using 5% EtOAc/hexane as a developer), thereby obtaining a yellow solid, Compound 3a (1.07 g, 68%).

¹H NMR (CDCl₃, 300 MHz, 298 K, δ): 7.79 (d, J=1.8 Hz, 1H), 7.52 (d, J=9.0 Hz, 1H), 7.48-7.45 (m, 1H), 7.42-7.38 (m, 1H), 6.86-6.82 (m, 1H), 6.74 (d, J=2.1 Hz, 1H), 3.79-3.70 (m, 2H), 1.28 (d, J=6.3 Hz, 6H); ¹³C NMR (CDCl₃, 75 MHz, 298 K, δ): 145.6, 134.0, 129.7, 129.6, 128.4, 128.2, 127.7, 119.3, 44.4, 23.0; IR (KBr, cm⁻¹): 2966, 1627, 1517; mp: 56-58° C.

### <2-2> Synthesis of Compound 3 (1-(6-(isopropylamino)naphthalen-2-yl)ethanone)

Compound 3, 1-(6-(isopropylamino)naphthalene-2-yl)ethanone, was synthesized by the inventors.

Specifically, Compound 3a obtained in Example 2-1 (550 mg, 2.1 mmol), Pd(OAc)_{2 (}23 mg, 0.11 mmol), DPPP (86 mg, 0.22 mmol), and ethyleneglycol (3 mL) were added to an oven-dried flask with two necks and charged with argon gas. Oxygen present in the mixture was removed by adding the argon gas to the mixture, and ethyleneglycolvinylether (1.14 mL, 6.2 mmol) and Et₃N obtained by distillation (723 μL, 5.2 mmol) were sequentially added thereto. The mixture was stirred at 145° C. for 5 hours using a silicone oil container. The mixture was cooled to room temperature and stirred with a 6N HCl aqueous solution (5 mL) at 60° C. for 4 hours. The mixture was cooled to room temperature and diluted with ethyl acetate (100 mL). An organic layer was washed with water (50 mL), a 5% sodium bicarbonate aqueous solution (50 mL), and a saturated saline solution (50 mL) and dehydrated with anhydrous sodium sulfate (10 g). The solvent was removed under a reduced pressure condition of 40 mbar, and the resulting product was purified by column chromatography through a silica gel (Merck-silicagel 60, 230-400 mesh; using 5% EtOAc/hexane as a developer), thereby obtaining a yellow solid, Compound 3 (322 mg, 68%). By further purification using recrystallization (using 4% CH₂Cl₂/hexane as a solvent), a yellow solid, Compound 3 (134 mg, 28%), was obtained.

¹H NMR (CDCl₃, 300 MHz, 298 K, δ): 8.26 (d, J=9.3 Hz, 1H), 7.91 (dd, J=8.7, 2.1 Hz, 1H), 7.71 (d, J=9.0 Hz, 1H), 7.56 (d, J=8.7 Hz, 1H), 6.86 (dd, J=9.0, 2.4 Hz, 1H), 6.76 (d, J=2.1 Hz, 1H), 3.94 (s, 1H), 3.83-3.75 (m, 1H), 2.66 (s, 3H), 1.3 (d, J=6.3 Hz, 6H); ¹³C NMR (CDCl₃, 75 MHz, 298 K, δ): 197.9, 147.6, 138.3, 131.0, 130.8, 130.6, 126.0, 125.8, 124.9, 119.0, 104.0, 44.2, 26.6, 22.9; IR (KBr, cm⁻¹): 1665; HRMS: m/z calcd for C₁₅H₁₇NO [M⁺] 227.1310, C₁₅H₁₈NO [MH⁺] 228.1388; found 227.1312 [M⁺], 228.1390 [M⁺H⁺]; mp: 112-114° C.

### Example 3

### Syntheses of Compounds 4 and 7

A general synthetic pathway of Compounds 4 and 7 is shown in Scheme 3.

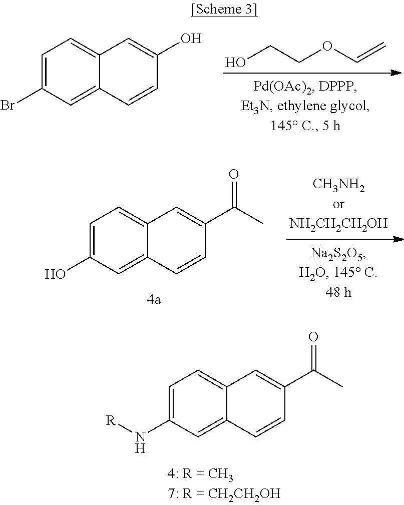

### <3-1> Synthesis of Compound 4a (1-(6-hydroxynaphthalen-2-yl)ethanone)

Compound 4a, 1-(6-hydroxynaphthalen-2-yl)ethanone, was synthesized by the inventors.

Specifically, starting materials for synthesis such as 6-bromo-2-naphthol (2.0 g, 8.97 mmol), Pd(OAc)₂ (100 mg, 0.45 mmol), DPPP (370 mg, 0.9 mmol), and ethylene glycol (3 mL) were added to an oven-dried flask with two necks and charged with argon gas. Oxygen present in the mixture was removed by adding the argon gas to the resulting mixture, and ethyleneglycolvinylether (2.41 mL, 27 mmol) and Et₃N obtained distillation (3.12 mL, 22.4 mmol) were sequentially added. The mixture was stirred at 145° C. for 4 hours using a silicone oil container. The mixture was cooled to room temperature and stirred with dichloromethane (15 mL) and a 5% HCl aqueous solution (30 mL) at room temperature for 1 hour. The resulting mixture was extracted with dichloromethane (2×30 mL), and an organic layer was washed with water (30 mL) and dehydrated with anhydrous sodium sulfate (6 g). The solvent was removed under a reduced pressure condition of 40 mbar, and the resulting product was purified by column chromatography through a silica gel (Merck-silicagel 60, 230-400 mesh; using CH₂Cl₂ as a developer), thereby obtaining a solid, Compound 4a (1.33 g, 80%).

¹H NMR (CDCl₃, 300 MHz, 298 K, δ): 8.41 (1H, s), 7.98 (1H, dd, J=8.7, 1.6 Hz), 7.87 (1H, d, J=8.7 Hz), 7.70 (1H, d, J=8.7 Hz), 7.16 (1H, dd, J=8.7, 1.6 Hz), 5.4 (1H, s), 2.71 (3H, s); mp 172° C.

### <3-2> Synthesis of Compound 4 (1-(6-(methylamino)naphthalen-2-yl)ethanone)

Compound 4, 1-(6-(methylamino)naphthalen-2-yl)ethanone, was synthesized by the inventors.

Specifically, water (20 mL) was added to a sealed tube containing Compound 4a obtained in Example 3-1 (2.0 g, 10.75 mmol), 50% methyl amine aqueous solution (4 mL, 53.75 mmol), and sodium metabisulfite (3.4 g, 21.5 mmol), and the tube was closed. The resulting mixture was stirred at 145° C. for 48 hours using a silicone oil container. The mixture was cooled to room temperature, and the resulting precipitate was filtered using a filter paper with a pore size of 8 μm and washed with water (10 mL). The filtered precipitate was purified by column chromatography through a silica gel (Merck-silicagel 60, 230-400 mesh; using 5% MeOH/CH₂Cl₂ as a developer), thereby obtaining a solid, Compound 4 (1.82 g, 85%).

¹H NMR (CDCl₃, 300 MHz, 298 K, δ): 8.3 (1H, s), 7.91 (1H, dd, J=8.7, 1.6 Hz), 7.70 (1H, d, J=8.7 Hz), 7.62 (1H, d, J=8.7 Hz), 6.89 (1H, dd, J=8.8, 2.2 Hz), 6.77 (1H, s), 4.17 (1H, br. s), 2.97 (3H, s), 2.67 (3H, s); mp 182° C.

### <3-3> Synthesis of Compound 7 (1-(6-(2-hydroxyethylamino)naphthalen-2-yl)ethanone)

Compound 7, 1-(6-(2-hydroxyethylamino)naphthalen-2-yl)ethanone, was synthesized by the inventors.

Specifically, water (15 mL) was added to a sealed tube containing Compound 4a obtained in Example 3-1 (1.0 g, 5.37 mmol), 2-aminoethanol (2-aminoethanol, 1.62 mL, 26.85 mmol), and sodium metabisulfite (2.0 g, 10.74 mmol), and the tube was closed. The resulting mixture was stirred at 145° C. for 48 hours using a silicone oil container. The mixture was cooled to room temperature, and the resulting precipitate was filtered using a filter paper with a pore size of 8 μm and washed with water (10 mL). The precipitated was purified by column chromatography through a silica gel (Merck-silicagel 60, 230-400 mesh; using 2% MeOH/CH₂Cl₂ as a developer), thereby obtaining a solid, Compound 7 (0.86 g, 70%).

¹H NMR (CDCl₃, 300 MHz, 298 K, δ): 8.31 (1H, s), 7.91 (1H, dd, J=9.0, 3.0 Hz, s), 7.72 (1H, d, J=9.0 Hz), 7.60 (1H, d, J=9.0 Hz), 6.94 (1H, dd, J=9.0 Hz), 6.84 (1H, s), 4.46 (1H, br. s), 3.94 (2H, t), 3.44 (2H, t), 2.67 (3H, s), 1.66 (1H, br. s); ¹³C NMR (CDCl₃+DMSO-d₆, 75 MHz, 298 K, δ): 197.74, 148.56, 138.05, 130.68, 130.63, 130.34, 125.87, 125.82, 124.60, 118.83, 103.45, 60.49, 45.75, 26.39; HRMS: m/z calcd for C₁₄H₁₅NO₂ [M⁺] 229.28; found 229.11 [M⁺].

### Example 4

### Syntheses of Compounds 5 and 9

A general synthetic pathway of Compounds 5 and 9 is shown in Scheme 4.

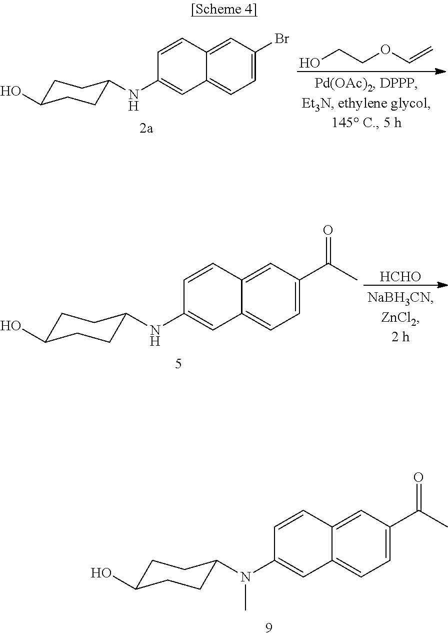

### <4-1> Synthesis of Compound 5 (1-(6-(4-hydroxycyclohexylamino)naphthalen-2-yl)ethanone)

Compound 5, 1-(6-(4-hydroxycyclohexylamino)naphthalen-2-yl)ethanone, was synthesized by the inventors.

Specifically, Compound 2a obtained in Example 1-1 (320 mg, 1.0 mmol), Pd(OAc)₂ (11.2 mg, 0.055 mmol), DPPP (45.39 mg, 0.11 mmol), and ethylene glycol (2 mL) were added to an oven-dried flask with two necks and charged with argon gas. Oxygen present in the resulting mixture was removed by adding the argon gas to the mixture, and ethyleneglycolvinylether (456 μL, 2.5 mmol) and Et₃N (417 μL, 3.0 mmol) obtained by distillation were sequentially added thereto. The mixture was stirred at 145° C. for 5 hours using a silicone oil container. The mixture was cooled to room temperature and stirred with a 6N HCl aqueous solution (2 mL) at 60° C. for 4 hours. The mixture was cooled to room temperature and diluted with ethyl acetate (150 mL). An organic layer was washed with water (50 mL), a 5% sodium bicarbonate aqueous solution (50 mL), and a saturated saline solution (50 mL) and dehydrated with anhydrous sodium sulfate (15 g). The solvent was removed under a reduced pressure condition of 40 mbar, and the resulting product was purified by column chromatography through a silica gel (Merck-silicagel 60, 230-400 mesh; using 50% EtOAc/CH₂Cl₂ as a developer), thereby obtaining a bright yellow solid, Compound 5 (203 mg, 72%). By further purification using recrystallization (using 7% CH₂Cl₂/hexane as a solvent), a bright yellow solid, Compound 5 (120 mg, 42%) was obtained.

¹H NMR (CD₃CN, 300 MHz, 298 K, δ): 8.33 (s, 1H), 7.84 (dd, J=8.7, 1.8 Hz, 1H), 7.73 (d, J=9.0 Hz, 1H), 7.59 (d, J=8.7 Hz, 1H), 6.96 (dd, J=9.0, 2.4 Hz, 1H), 6.83 (d, J=1.8 Hz, 1H), 4.84 (d, J=7.8 Hz, 1H), 3.62-3.5 (s, 1H), 3.46-3.34 (m, 1H), 2.71 (d, J=4.5 Hz, 1H), 2.59 (s, 3H), 2.13-2.09 (m, 4H), 1.46-1.2 (m, 4H); ¹H NMR (DMSO-d₆, 500 MHz, δ): 8.33 (d, J=1 Hz, 1H), 7.77-7.73 (m, 2H), 7.58 (d, J=8.5 Hz, 1H), 7.02 (dd, J=9.0, 2.0 Hz, 1H), 6.76 (d, J=1.5 Hz, 1H), 6.21 (d, J=7.5 Hz, 1H), 4.57 (d, J=4.5 Hz, 1H), 3.49-3.43 (m, 1H), 3.35-3.3 (m, 1H), 2.58 (s, 3H), 2.03-2 (m, 2H), 1.89-1.86 (m, 2H), 1.38-1.31 (m, 2H), 1.27-1.2 (m, 2H); ¹³C NMR (CDCl₃, DMSO-d₆, 125 MHz, 300K, δ): 196.8, 148.3, 138.0, 130.4, 129.4, 125.2, 124.6, 124.0, 119.0, 102.0, 68.4, 50.1, 33.9, 30.1, 26.3; IR (KBr, cm⁻¹): 1669; HRMS: m/z calcd for C₁₈H₂₁NO₂ [M⁺] 283.1572, C₁₈H₂₂NO₂ [MH⁺] 284.1651; found 283.1575 [M⁺], 284.1648 [MH⁺]; mp: 186-188° C.

### <4-2> Synthesis of Compound 9 (1-(6-((4-hydroxycyclohexyl)(methyl)amino)naphthalen-2-yl)ethanone)

Compound 9, 1-(6-((4-hydroxycyclohexyl)(methyl)amino)naphthalen-2-yl)ethanone, was synthesized by the inventors.

Specifically, Compound 5 obtained in Example 4-1 (50 mg, 0.176 mmol) was dissolved in methanol (5 mL) and stirred in a 37% formaldehyde aqueous solution (43 μL, 0.53 mmol). A solution prepared by dissolving sodium cyanoborohydride (11.1 mg, 0.176 mmol) and zinc chloride (12 mg, 0.088 mmol) in methanol (2 mL) was added to the resulting mixture and stirred at room temperature for 2 hours (the reaction progress was checked by TLC). An 0.1N sodium hydroxide (NaOH) aqueous solution (2 mL) was added to the mixture, methanol was removed under a reduced pressure condition of 40 mbar, and then extraction with ethyl acetate (3×10 mL) was performed. An organic layer was washed with water (10 mL) and a saturated saline solution (10 mL) and dehydrated with anhydrous magnesium sulfate (MgSO₄, 3 g). The solvent was removed under a reduced pressure condition of 40 mbar, and the resulting product was purified by column chromatography through a silica gel (Merck-silicagel 60, 230-400 mesh; using 50% EtOAc/hexane as a developer), thereby obtaining a bright yellow solid, Compound 9 (43 mg, 81%). By further purification using recrystallization (using 5% CH₂Cl₂/hexane as a solvent), a bright yellow solid, Compound 9 (27 mg, 45%) was obtained.

¹H NMR (CDCl₃, 300 MHz, 298 K, δ): 8.31 (s, 1H), 7.90 (dd, J=8.7, 1.8 Hz, 1H), 7.78 (d, J=9.3 Hz, 1H), 7.61 (d, J=9.0 Hz, 1H), 6.19 (dd, J=9.3, 2.4 Hz, 1H), 6.91 (d, J=2.1 Hz, 1H), 3.87-3.77 (m, 1H), 3.71-3.66 (m, 1H), 2.91 (s, 3H), 2.67 (s, 3H), 2.17-2.02 (m, 2H), 1.88-1.80 (m, 2H), 1.73-1.37 (m, 4H); ¹³C NMR (CDCl₃, 75 MHz, 300 K, δ): 198.0, 150.2, 138.0, 131.1, 131.0, 130.5, 126.4, 124.9, 117.1, 106.3, 70.4, 57.4, 35.1, 31.6, 29.9, 27.9, 26.6; IR (KBr, cm⁻¹): 1672; HRMS: m/z calcd for C₁₉H₂₃NO₂ [M⁺] 297.1729, C₁₉H₂₄NO₂ [MH⁺] 298.1761; found 297.1727 [M⁺], 297.1766 [MH⁺]; mp: 192-194° C.

### Example 5

### Synthesis of Compound 6

A general synthetic pathway of Compound 6 is shown in Scheme 5.

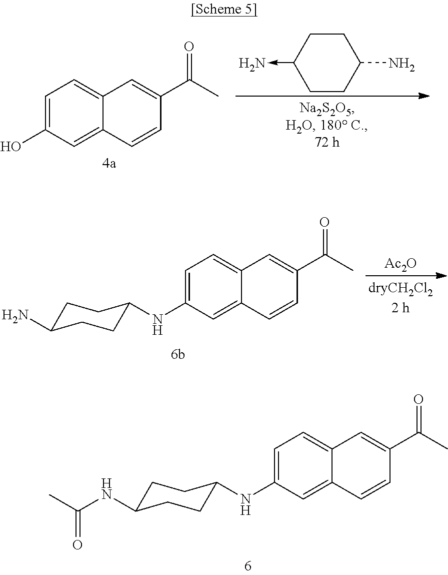

### <5-1> Synthesis of Compound 6b (1-(6-(4-aminocyclohexylamino)naphthalen-2-yl)ethanone)

Compound 6b, 1-(6-(4-aminocyclohexylamino)naphthalen-2-yl)ethanone, was synthesized by the inventors.

Specifically, water (10 mL) was added to a sealed tube containing Compound 4a obtained in Example 3-1 (418 mg, 1.68 mmol), trans-1,4-diaminocyclohexane (383 mg, 3.36 mmol), and sodium metabisulfite (640 mg, 3.36 mmol), and the tube was closed. The resulting mixture was stirred at 180° C. for 72 hours using a silicone oil container. The mixture was cooled to room temperature (25° C.) and filtered using cotton. Following removal of the solvent under a reduced pressure condition of 40 mbar, the filtered liquid was purified by column chromatography through a silica gel (Merck-silicagel 60, 230-400 mesh; using 5% MeOH/CH₂Cl₂ as a developer), thereby obtaining a brown solid, Compound 6b (355 mg, 56%). By further purification using recrystallization (using 25% MeOH/CH₂Cl₂ as a solvent), a brown solid, Compound 6b (139 mg, 22%) was obtained.

¹H NMR (CD₃OD, 300 MHz, 298 K, δ): 8.34 (d, J=1.5 Hz, 1H), 7.82 (dd, J=8.7, 1.8 Hz, 1H), 7.71 (d, J=9.0 Hz, 1H), 7.56 (d, J=8.7 Hz, 1H), 6.97 (dd, J=9.0, 2.4 Hz, 1H), 6.80 (d, J=2.1 Hz, 1H), 3.46-3.41 (m, 1H), 3.08-3.02 (m, 1H), 2.64 (s, 3H), 2.25-2.21 (m, 2H), 2.11-2.07 (m, 2H), 1.53-1.43 (m, 2H), 1.42-1.30 (m, 2H); ¹³C NMR (CD₃OD, 75 MHz, 298 K, δ): 200.6, 149.9, 140.2, 132.2, 132.1, 131.4, 127.1, 126.9, 125.4, 120.3, 104.3, 51.6, 51.2, 32.1, 31.9, 26.5; IR (KBr, cm⁻¹): 3321, 1668, 1550; mp: 198-200° C.

### <5-2> Synthesis of Compound 6 (N-(4-(6-acetylnaphthalen-2-ylamino)cyclohexyl)acetamide)

Compound 6, N-(4-(6-acetylnaphthalen-2-ylamino)cyclohexyl)acetamide, was synthesized by the inventors.

Specifically, compound 6b obtained in Example 5-1 (283 mg, 1.0 mmol) was dissolved in anhydrous dichloromethane (50 mL), and a solution prepared by dissolving acetic anhydride (94 μL, 1.0 mmol) in anhydrous dichloromethane (10 mL) was added to the resulting mixture. The mixture was stirred at room temperature for 2 hours, and a saturated ammonium chloride (NH₄Cl) aqueous solution (10 mL) was added. An organic layer was washed with water (10 mL) and a saturated saline solution (10 mL) and dried with anhydrous magnesium sulfate (6 g). The solvent was removed under a reduced pressure condition of 40 mbar, the resulting product was purified by column chromatography through a silica gel (Merck-silicagel 60, 230-400 mesh; using 5% MeOH/CH₂Cl₂ as a developer), thereby obtaining a brown solid, Compound 6 (299 mg, 92%). By further purification using recrystallization (using 5% MeOH/CH₂Cl₂ as a solvent), a brown solid, Compound 6 (125 mg, 38%), was obtained.

¹H NMR (CDCl₃, 300 MHz, 298 K, δ): 8.27 (s, 1H), 7.9 (dd, J=8.7, 1.8 Hz, 1H), 7.69 (d, J=8.7 Hz, 1H), 7.57 (d, J=8.7 Hz, 1H), 6.84 (dd, J=8.7, 2.4 Hz, 1H), 6.74 (d, J=1.8 Hz, 1H), 5.36 (d, J=8.1 Hz, 1H), 3.98 (br, 1H), 3.85-3.83 (m, 1H), 3.39 (br, 1H), 2.66 (s, 3H), 2.25-2.23 (m, 2H), 2.11-2.08 (m, 2H), 1.99 (s, 3H), 1.37-1.30 (m, 4H); ¹³C NMR (CDCl₃, 75 MHz, 298 K, δ): 198.0, 169.6, 147.3, 138.2, 131.2, 131.1, 130.6, 126.0, 125.0, 118.9, 104.1, 51.3, 48.2, 32.1, 32.0, 26.6, 23.8; IR (KBr, cm⁻¹): 1653, 1576; HRMS: m/z calcd for C₂₀H₂₄N₂O₂ [M⁺] 324.1838, [M⁺] 325.1869; found 324.1835 [M⁺], 325.1871 [M⁺]; mp: above 250° C.

### Example 6

### Synthesis of Compound 8

A general synthetic pathway of Compound 8 is shown in Scheme 6.

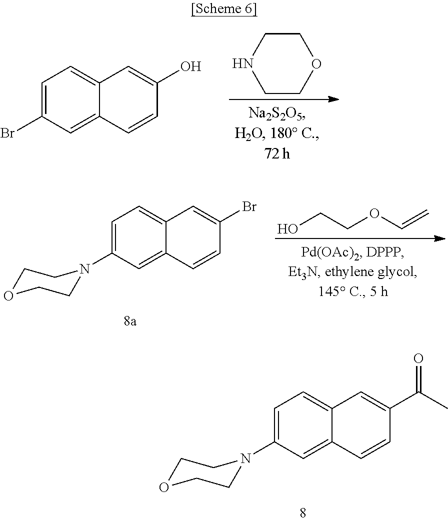

### <6-1> Synthesis of Compound 8a (4-(6-bromonaphthalen-2-yl)morpholine)

Compound 8a, 4-(6-bromonaphthalen-2-yl)morpholine, was synthesized by the inventors.

Specifically, water (15 mL) was added to a sealed tube containing starting materials for synthesis such as 6-bromo-2-naphthol (1.5 g, 6.72 mmol), morpholine (morpholine, 2.93 g, 33.60 mmol), and sodium metabisulfite (2.56 g, 13.45 mmol), and the tube was closed. The resulting mixture was stirred at 180° C. for 72 hours using a silicone oil container. The mixture was cooled to room temperature and diluted with ethyl acetate (300 mL) following opening of the tube. An organic layer was washed with water (80 mL), a 5% sodium bicarbonate aqueous solution (50 mL), and a saturated saline solution (50 mL), and dehydrated with anhydrous sodium sulfate (30 g). The solvent was removed under a reduced pressure condition of 40 mbar, and the resulting product was purified by column chromatography through a silica gel (Merck-silicagel 60, 230-400 mesh; using 1% MeOH/CH₂Cl₂ as a developer), thereby obtaining a brown solid, Compound 8a (1.21 g, 62%).

¹H NMR (CDCl₃, 300 MHz, 298 K, δ): 7.87 (d, J=1.8 Hz, 1H), 7.64 (d, J=9.3 Hz, 1H), 7.55 (d, J=9.0 Hz, 1H), 7.46 (dd, J=8.7, 2.1 Hz, 1H), 7.24-7.28 (m, 1H), 7.06 (m, 1H), 3.89-3.95 (m, 4H), 3.24-3.30 (m, 4H); ¹³C NMR (CDCl₃, 75 MHz, 298 K, δ): 149.6, 133.2, 129.8, 129.7, 128.6, 128.2, 119.87, 117.1, 110.0, 67.1, 49.7; IR (KBr, cm⁻¹): 1617, 1570; mp: 158-160° C.

### <6-2> Synthesis of Compound 8 (1-(6-morpholinonaphthalen-2-yl)ethanone)

Compound 8, 1-(6-morpholinonaphthalen-2-yl)ethanone, was synthesized by the inventors.

Specifically, Compound 8a obtained in Example 6-1 (97 mg, 0.33 mmol), Pd(OAc)₂ (3.8 mg, 0.017 mmol), DPPP (13.8 mg, 0.034 mmol), and ethylene glycol (1 mL) were added to an oven-dried flask with two necks and charged with argon gas. Oxygen present in the resulting mixture was removed by adding the argon gas to the mixture, and ethyleneglycolvinylether (183 μL, 1.0 mmol) and Et₃N (116 μL, 0.84 mmol) obtained by distillation were sequentially added thereto. The mixture was stirred at 145° C. for 4 hours using a silicone oil container. The mixture was cooled to room temperature and stirred with a 6N HCl aqueous solution (1.5 mL) at 60° C. for 4 hours. The mixture was cooled to room temperature and diluted with ethyl acetate (100 mL). An organic layer was washed with water (3×50 mL) and a saturated saline solution (50 mL) and dehydrated with anhydrous sodium sulfate (10 g). The solvent was removed under a reduced pressure condition of 40 mbar, and the resulting product was purified by column chromatography through a silica gel (Merck-silicagel 60, 230-400 mesh; using 1% MeOH/CH₂Cl₂ as a developer), thereby obtaining a brown solid, Compound 8 (54 mg, 64%). By further purification using recrystallization (using 1% MeOH/CH₂Cl₂ as a solvent), a brown solid, Compound 8 (36 mg, 43%), was obtained.

¹H NMR (CDCl₃, 300 MHz, 298 K, δ): 8.34 (s, 1H), 7.97 (d, J=8.7 Hz, 1H), 7.84 (d, J=9.3 Hz, 1H), 7.69 (d, J=8.7 Hz, 1H), 7.26-7.31 (m, 1H), 7.1 (s, 1H), 3.91 (t, J=4.8 Hz, 1H), 3.32 (t, J=4.6 Hz, 1H), 2.68 (s, 3H); ¹³C NMR (CDCl₃, 75 MHz, 298 K, δ): 198.0, 151.2, 137.4, 132.4, 130.9, 130.2, 127.3, 127.1, 124.9, 119.0, 109.2, 67.0, 49.0, 26.7; IR (KBr, cm⁻¹): 1666; HRMS: m/z calcd for C₁₆H₁₇NO₂ [M⁺] 255.1259, C₁₆H₁₈NO₂ [MH⁺] 256.1292; found 255.1256 [M⁺], 256.1279 [MH⁺]; mp: 149-151° C.

### Example 7

### Synthesis of Compound 10 (6-(4-hydroxycyclohexylamino)-2-(2-hydroxyethyl)-1H-benzo[de]isoquinoline-1,3(2H)-dione)

A general synthetic pathway of Compound 10 is shown in Scheme 7.

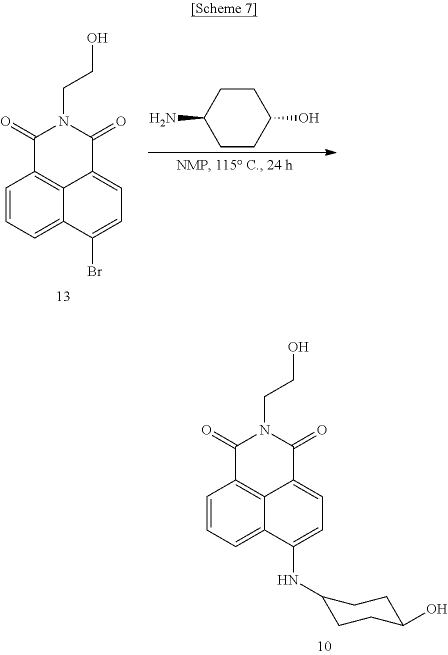 [SMILES: O=C1c2cccc3c(Br)ccc(c23)C(=O)N1CCO ; N[C@H]1CC[C@H](O)CC1 ; O=C1c2cccc3c(NC4CCC(O)CC4)ccc(c23)C(=O)N1CCO]

Compound 10, 6-(4-hydroxycyclohexylamino)-2-(2-hydroxyethyl)-1H-benzo[de] isoquinoline-1,3(2H)-dione, was synthesized by the inventors. Specifically, N-methyl pyrrolidone (NMP, 2 mL) was added to a sealed tube containing known starting materials for synthesis such as Compound 13 (S. Ghorbanian, S. et al. J. Chem. Technol. Biotechnol. 75, 1127.; 320 mg, 1.0 mmol) and trans-4-aminocyclohexanol (230 mg, 2.0 mmol), and the tube was closed. The resulting mixture was stirred at 115° C. for 24 hours using a silicone oil container. The mixture was cooled to room temperature and diluted with ethyl acetate (200 mL). An organic layer was washed with water (50 mL) and a saturated saline solution (50 mL) and dehydrated with anhydrous sodium sulfate (20 g). After the solvent was removed under a reduced pressure condition of 40 mbar, hexene (20 mL) was slowly added to the resulting product dissolved in chloroform (2 mL), thereby obtaining a yellow precipitate. The precipitate was filtered using a filter paper with a pore size of 8 μm and washed with water (10 mL) and hexene (10 mL). The precipitate was purified by column chromatography through a silica gel (Merck-silicagel 60, 230-400 mesh; using EtOAc as a developer), thereby obtaining an orange solid, Compound 10 (205 mg, 64%).

¹H NMR (DMSO-d₆, 300 MHz, 298 K, δ): 8.75 (d, J=8.1 Hz, 1H), 8.42 (d, J=6.9 Hz, 1H), 8.24 (d, J=8.4 Hz, 1H), 7.64 (t, J=7.5 Hz, 1H), 7.23 (d, J=7.8 Hz, 1H), 6.85 (d, J=9.0 Hz, 1H), 4.76 (t, J=6.0 Hz, 1H), 4.63 (d, J=4.2 Hz, 1H), 4.10 (t, J=6.6 Hz, 1H), 3.61-3.57 (m, 3H), 3.61-3.57 (m, 1H), 2.00 (d, J=11.7 Hz, 2H), 1.89 (d, J=11.7 Hz, 2H), 1.35-52 (m, 4H); ¹³C NMR (DMSO-d₆, 125 MHz, 298 K, δ): 164.4, 163.5, 150.3, 034.7, 131.1, 130.1, 129.3, 124.5, 122.4, 120.6, 108.0, 104.7, 68.9, 58.5, 51.5, 41.8, 34.5, 30.2; HRMS: m/z calcd for C₂₀H₂₃N₂O₄ [MH⁺] 355.1658; found 355.1659 [MH⁺]; mp: above 250° C.

### Example 8

### Synthesis of Compound 13 (1-6-(((1S,2S)-2-hydroxycyclohexyl)amino)naphthalen-2-yl)ethanone)

A general synthetic pathway of Compound 13 is shown in Scheme 8.

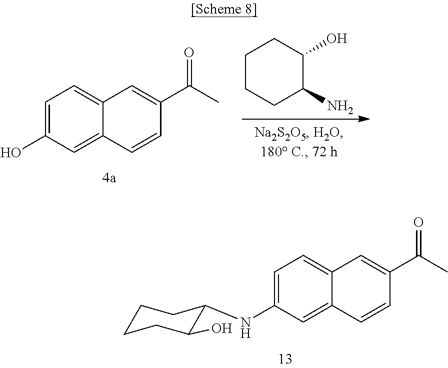

Compound 13, 1-6-(((1S,2S)-2-hydroxycyclohexyl)amino)naphthalen-2-yl)ethanone, was synthesized by the inventors. Specifically, water (15 mL) was added to a sealed tube containing Compound 4a obtained in Example 3-1 (1.25 g, 6.73 mmol), sodium metabisulfite (2.56 g, 13.45 mmol), and (1S,2S)-2-aminocyclohexanol (3.87 g, 33.65 mmol), and the tube was closed. The resulting mixture was stirred at 180° C. for 72 hours using a silicone oil container. After the mixture was cooled to room temperature, the container was opened to dilute the mixture with ethyl acetate (300 mL). An organic layer was washed with water (80 mL), a 5% sodium bicarbonate aqueous solution (50 mL), and a saturated saline solution (50 mL) and dehydrated with anhydrous sodium sulfate (30 g). The solvent was removed under a reduced pressure condition of 40 mbar, and the resulting product was purified by column chromatography through a silica gel (Merck-silicagel 60, 230-400 mesh; using 30% EtOAc/hexane as a developer), thereby obtaining a solid, Compound 13 (1.18 g, 62%).

¹H NMR (CDCl₃, 300 MHz, 298 K, δ): 8.18 (s, 1H), 7.83 (dd, J=8.7, 1.8 Hz, 1H), 7.58 (d, J=9.0 Hz, 1H), 7.48 (d, J=8.7 Hz, 1H), 6.84 (dd, J=8.7, 2.1 Hz, 1H), 6.72 (d, J=1.8 Hz, 1H), 4.61 (br, s, 1H), 4.13 (br, s, 1H), 3.533.50 (m, 1H), 2.92 (d, J=3.3 Hz, 1H), 2.59 (s, 3H), 1.90-1.86 (m, 1H), 1.781.58 (m, 5H), 1.471.37 (m, 2H); ¹³C NMR (CDCl₃, 75 MHz, 298 K, δ): 198.3, 147.5, 138.1, 130.9, 130.6, 130.5, 125.8, 125.7, 124.6, 119.0, 104.1, 67.7, 54.3, 31.8, 26.7, 26.4, 24.1, 20.0.

### Example 9

### Synthesis of Compound 14 (1-6-(((1S,2R)-2-hydroxycyclohexyl)amino)naphthalen-2-yl)ethanone)

A general synthetic pathway of Compound 14 is shown in Scheme 9.

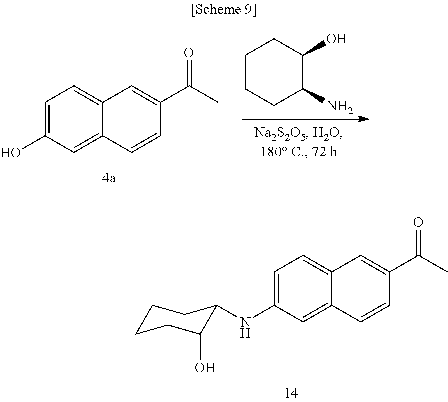

Compound 13, 1-6-(((1 S,2R)-2-hydroxycyclohexyl)amino)naphthalen-2-yl)ethanone, was synthesized by the inventors. Specifically, water (15 mL) was added to a sealed tube containing Compound 4a obtained in Example 3-1 (1.25 g, 6.73 mmol), sodium metabisulfite (2.56 g, 13.45 mmol), and (1S,2R)-2-aminocyclohexanol (3.87 g, 33.65 mmol), and the tube was closed. The mixture was stirred at 180° C. for 72 hours using a silicone oil container. After the mixture was cooled to room temperature, the container was opened to dilute the mixture with ethyl acetate (300 mL). An organic layer was washed with water (80 mL), a 5% sodium bicarbonate aqueous solution (50 mL), and a saturated saline solution (50 mL) and dehydrated with anhydrous sodium sulfate (30 g). The solvent was removed under a reduced pressure condition of 40 mbar, and the resulting product was purified by column chromatography through a silica gel (Merck-silicagel 60, 230-400 mesh; using 30% EtOAc/hexane as a developer), thereby obtaining a solid, Compound 14 (1.18 g, 62%).

¹H NMR (CDCl₃, 300 MHz, 298 K, δ): 8.18 (s, 1H), 7.83 (dd, J=8.7, 1.8 Hz, 1H), 7.58 (d, J=9.0 Hz, 1H), 7.48 (d, J=8.7 Hz, 1H), 6.84 (dd, J=8.7, 2.1 Hz, 1H), 6.72 (d, J=1.8 Hz, 1H), 4.61 (br, s, 1H), 4.13 (br, s, 1H), 3.533.50 (m, 1H), 2.92 (d, J=3.3 Hz, 1H), 2.59 (s, 3H), 1.90-1.86 (m, 1H), 1.781.58 (m, 5H), 1.471.37 (m, 2H); ¹³C NMR (CDCl₃, 75 MHz, 298 K, δ): 198.3, 147.5, 138.1, 130.9, 130.6, 130.5, 125.8, 125.7, 124.6, 119.0, 104.1, 67.7, 54.3, 31.8, 26.7, 26.4, 24.1, 20.0.

### Example 10

### Synthesis of Compound 15

A general synthetic pathway of Compound 15 is shown in Scheme 10.

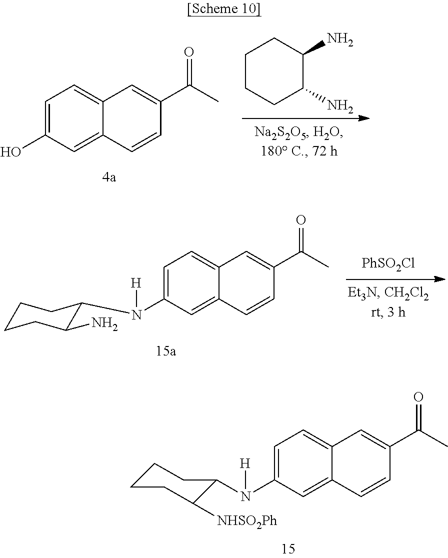

### <10-1> Synthesis of Compound 15a (1-(6-(1S,2S)-2-aminocyclohexyl)amino)naphthalen-2-yl)ethanone)

Compound 15a, 1-(6-(((1S,2S)-2-aminocyclohexyl)amino)naphthalen-2-yl)ethanone, was synthesized by the inventors. Specifically, water (15 mL) was added to a sealed tube containing Compound 4a obtained in Example 3-1 (1.25 g, 6.73 mmol), sodium metabisulfite (2.56 g, 13.45 mmol), and (1R,2R)-cyclohexane-1,2-diamine (1.53 g, 13.46 mmol), and the tube was closed. The resulting mixture was stirred at 180° C. for 72 hours using a silicone oil container. After the mixture was cooled to room temperature, the container was opened to dilute the mixture with ethyl acetate (300 mL). An organic layer was washed with water (80 mL), 5% sodium bicarbonate aqueous solution (50 mL) and a saturated saline solution (50 mL), and dehydrated with anhydrous sodium sulfate (30 g). The solvent was removed under a reduced pressure condition of 40 mbar, and the resulting product was purified by column chromatography through a silica gel (Merck-silicagel 60, 230-400 mesh; using 30% EtOAc/hexane as a developer), thereby obtaining a solid, Compound 14 (1.27 g, 67%).

¹H NMR (CDCl₃, 300 MHz, 298 K, δ): 8.19 (s, 1H), 7.83 (d, J=8.7 Hz, 1H), 7.59 (d, J=8.7 Hz, 1H), 7.50 (d, J=8.7 Hz, 1H), 6.92 (d, J=8.1 Hz, 1H), 6.80 (s, 1H), 4.38 (br, s, 1H), 3.873.43 (br, 3H), 3.23-3.08 (m, 1H), 2.59 (s, 3H), 2.152.11 (m, 1H), 2.011.94 (m, 1H), 1.721.68 (m, 2H), 1.391.10 (m, 3H), 1.09-0.89 (m, 1H).

### <10-2> Synthesis of Compound 15 (N-((2S)-2-((6-acetylnaphthalen-2-yl)amino)cyclohexyl)benzenesulfonamide)

Compound 15, N-((2S)-2-((6-acetylnaphthalen-2-yl)amino)cyclohexyl)benzenesulfonamide, was synthesized by the inventors. Specifically, Compound 15a obtained in Example 10-1 (93 mg, 0.33 mmol), benzenesulfonyl chloride (64 mg, 0.36 mmol), and triethylamine (36 mg, 0.36 mmol) were added to a flask and charged with argon gas. The resulting mixture was dissolved in dichloromethane, stirred at room temperature for 3 hours, and then diluted with dichloromethane (100 mL). An organic layer was washed with water (3×50 mL) and a saturated saline solution (50 mL) and dehydrated with anhydrous sodium sulfate (10 g). The solvent was removed under a reduced pressure condition of 40 mbar, and the resulting product was purified by column chromatography through a silica gel (Merck-silicagel 60, 230-400 mesh; using 1% MeOH/CH₂Cl₂ as a developer), thereby obtaining a solid, Compound 15 (120 mg, 86%).

¹H NMR (CDCl₃, 300 MHz, 298 K, δ): 8.27 (d, J=1.2 Hz, 1H), 7.937.85 (m, 3H), 7.66 (d, J=8.7 Hz, 1H), 7.607.54 (m, 2H), 7.507.45 (m, 2H), 6.73 (dd, J=8.7, 2.1 Hz, 1H), 6.67 (d, J=2.1 Hz, 1H), 4.87 (d, J=6.6 Hz, 1H), 4.23 (d, J=7.2 Hz, 1H), 3.253.18 (m, 1H), 3.153.08 (m, 1H), 2.66 (s, 3H), 2.33-2.29 (m, 1H), 1.931.89 (m, 1H), 1.751.65 (m, 2H), 1.391.23 (m, 3H), 1.191.12 (m, 1H); ¹³C NMR (CDCl₃, 75 MHz, 298 K, δ): 198.1, 147.4, 140.9, 138.1, 132.9, 131.1, 131.1, 130.6, 129.4, 127.1, 126.2, 126.1, 124.9, 119.1, 104.0, 57.1, 56.7, 33.4, 32.1, 26.6, 24.8, 24.2.

### Example 11

### Synthesis of Compound 16

A general synthetic pathway of Compound 16 is shown in Scheme 11.

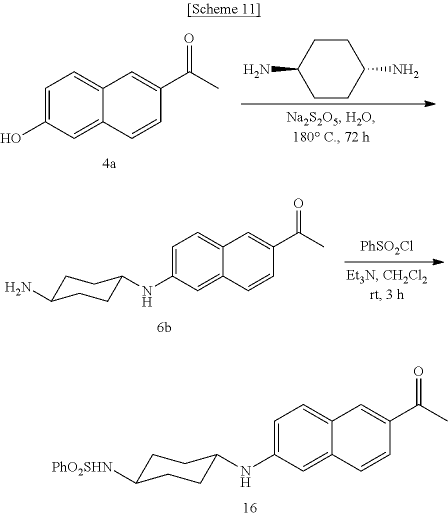

### <11-1> Synthesis of Compound 6b (1-(6-(((1R,4R)-4-aminocyclohexyl)amino)naphthalen-2-yl)ethanone)

Compound 15a, 1-(6-(((1S,2S)-2-aminocyclohexyl)amino)naphthalen-2-yl)ethanone, was synthesized by the inventors. Specifically, water (10 mL) was added to a sealed tube containing Compound 4a obtained in Example 3-1 (418 mg, 1.68 mmol), trans-1,4-diaminocyclohexane (383 mg, 3.36 mmol), and sodium metabisulfite (640 mg, 3.36 mmol), and the tube was closed. The mixture was stirred at 180° C. for 72 hours using a silicone oil container. The mixture was cooled to room temperature (25° C.) and filtered using cotton. After the solvent was removed under a reduced pressure condition of 40 mbar, the filtered liquid was purified by column chromatography through a silica gel (Merck-silicagel 60, 230-400 mesh; using 5% MeOH/CH₂Cl₂ as a developer), thereby obtaining a brown solid, Compound 6b (355 mg, 56%). By further purification using recrystallization (using 25% MeOH/CH₂Cl₂ as a solvent), a brown solid, Compound 6b (139 mg, 22%) was obtained.

¹H NMR (CD₃OD, 300 MHz, 298 K, δ): 8.34 (d, J=1.5 Hz, 1H), 7.82 (dd, J=8.7, 1.8 Hz, 1H), 7.71 (d, J=9.0 Hz, 1H), 7.56 (d, J=8.7 Hz, 1H), 6.97 (dd, J=9.0, 2.4 Hz, 1H), 6.80 (d, J=2.1 Hz, 1H), 3.46-3.41 (m, 1H), 3.08-3.02 (m, 1H), 2.64 (s, 3H), 2.25-2.21 (m, 2H), 2.11-2.07 (m, 2H), 1.53-1.43 (m, 2H), 1.42-1.30 (m, 2H); ¹³C NMR (CD₃OD, 75 MHz, 298 K, δ): 200.6, 149.9, 140.2, 132.2, 132.1, 131.4, 127.1, 126.9, 125.4, 120.3, 104.3, 51.6, 51.2, 32.1, 31.9, 26.5; IR (KBr, cm⁻¹): 3321, 1668, 1550; mp: 198-200° C.

### <11-2> Synthesis of Compound 16 (N-((1R,4R)-4-((6-acetylnaphthalen-2-yl)amino)cyclohexyl)benzenesulfonamide)

Compound 16, N-((1R,4R)-4-((6-acetylnaphthalen-2-yl)amino)cyclohexyl)benzenesulfonamide, was synthesized by the inventors. Specifically, Compound 6b obtained in Example 11-1 (93 mg, 0.33 mmol), benzenesulfonyl chloride (64 mg, 0.36 mmol), and triethylamine (36 mg, 0.36 mmol) were added to a flask and then charged with argon gas, and then the resulting mixture was dissolved with dichloromethane. The mixture was stirred at room temperature for 3 hours and diluted with dichloromethane (100 mL). An organic layer was washed with water (3×50 mL) and a saturated saline solution (50 mL) and dehydrated with anhydrous sodium sulfate (10 g). The solvent was removed under a reduced pressure condition of 40 mbar, and the resulting product was purified by column chromatography through a silica gel (Merck-silicagel 60, 230-400 mesh; using 1% MeOH/CH₂Cl₂ as a solvent), thereby obtaining a solid, Compound 15 (120 mg, 86%).

¹H NMR (CDCl₃, 300 MHz, 298 K, δ): 8.26 (d, J=1.2 Hz, 1H), 7.94-7.89 (m, 3H), 7.67 (d, J=8.7 Hz, 1H), 7.607.50 (m, 4H), 6.80 (dd, J=9.0, 2.4 Hz, 1H), 6.69 (d, J=2.1 Hz, 1H), 4.794.77 (m, 1H), 3.89 (br, s, 1H), 3.333.18 (m, 2H), 2.65 (s, 3H), 2.172.13 (m, 2H), 1.971.93 (m, 2H), 1.441.31 (m, 2H), 1.271.14 (m, 2H); ¹³C NMR (CDCl₃, 75 MHz, 298 K, δ): 198.0, 147.1, 141.4, 138.1, 131.2, 131.1, 129.4, 127.1, 126.0, 125.0, 118.8, 104.1, 52.6, 50.7, 32.8, 31.7, 26.6.

### Example 12

### Synthesis of Compound 17 (1-6(cyclohexylamino)naphthalen-2-yl)ethanone)

A general synthetic pathway of Compound 17 is shown in Scheme 12.

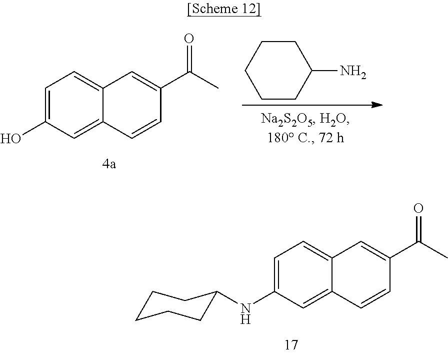

Compound 17, 1-6-(cyclohexylamino)naphthalen-2-yl)ethanone, was synthesized by the inventors. Specifically, water (15 mL) was added to a sealed tube containing Compound 4a obtained in Example 3-1 (1.25 g, 6.73 mmol), sodium metabisulfite (2.56 g, 13.45 mmol), and cyclohexaneamine (3.33 g, 33.65 mmol), and the tube was closed. The mixture was stirred at 180° C. for 72 hours using a silicone oil container. The mixture was cooled to room temperature and diluted with ethyl acetate (300 mL). An organic layer was washed with water (80 mL), a 5% sodium bicarbonate aqueous solution (50 mL), and a saturated saline solution (50 mL) and dehydrated with anhydrous sodium sulfate (30 g). The solvent was removed under a reduced pressure condition of 40 mbar, and the resulting product was purified by column chromatography through a silica gel (Merck-silicagel 60, 230-400 mesh; using 30% EtOAc/hexane as a developer), thereby obtaining a solid, Compound 17 (1.25 g, 70%).

¹H NMR (CDCl₃, 300 MHz, 298 K, δ): 8.27 (d, J=1.5 Hz, 1H), 7.89 (dd, J=8.7, 1.8 Hz, 1H), 7.69 (d, J=8.7 Hz, 1H), 7.57 (d, J=8.7 Hz, 1H), 6.85 (dd, J=9.0, 2.4 Hz, 1H), 6.76 (d, J=2.1 Hz, 1H), 4.00 (br, s, 1H), 3.463.39 (m, 1H), 2.66 (s, 3H), 2.152.11 (m, 2H), 1.841.78 (m, 2H), 1.841.78 (m, 2H), 1.69-1.61 (m, 1H), 1.481.38 (m, 2H), 1.321.21 (m, 3H); ¹³C NMR (CDCl₃, 75 MHz, 298 K, δ): 197.9, 147.5, 138.3, 131.1, 130.8, 130.6, 126.0, 125.8, 124.9, 118.9, 103.9, 51.6, 33.3, 26.6, 26.0, 25.1.

### Example 13

### Synthesis of Compound 18 (1-6-(pyrrolidin-1-yl)naphthalen-2-yl)ethanone)

A general synthetic pathway of Compound 18 is shown in Scheme 13.

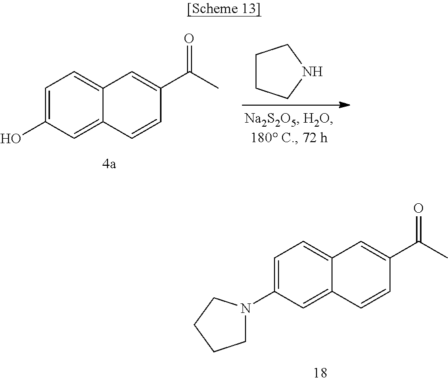

Compound 18, 1-6-(pyrrolidin-1-yl)naphthalen-2-yl)ethanone, was synthesized by the inventors. Specifically, water (15 mL) was added to a sealed tube containing Compound 4a obtained in Example 3-1 (1.25 g, 6.73 mmol), sodium metabisulfite (2.56 g, 13.45 mmol), and pyrrolidine (2.59 g, 33.65 mmol), and the tube was closed. The mixture was stirred at 180° C. for 72 hours using a silicone oil container. After the mixture was cooled to room temperature, the container was opened to dilute the mixture with ethyl acetate (300 mL). An organic layer was washed with water (80 mL), a 5% sodium bicarbonate aqueous solution (50 mL), and a saturated saline solution (50 mL) and dehydrated with anhydrous sodium sulfate (30 g). The solvent was removed under a reduced pressure condition of 40 mbar, and the resulting product was purified by column chromatography through a silica gel (Merck-silicagel 60, 230-400 mesh; using 30% EtOAc/hexane as a developer), thereby obtaining a solid, Compound 18 (1.15 g, 72%).

¹H NMR (CDCl₃, 300 MHz, 298 K, δ): 8.29 (d, J=0.9 Hz, 1H), 7.89 (dd, J=8.7, 1.8 Hz, 1H), 7.74 (d, J=9.0 Hz, 1H), 7.57 (d, J=8.7 Hz, 1H), 6.96 (dd, J=9.0, 2.4 Hz, 1H), 6.69 (d, J=2.1 Hz, 1H), 3.38 (t, J=6.6 Hz, 4H), 2.65 (s, 3H), 2.092.00 (m, 4H); ¹³C NMR (CDCl₃, 75 MHz, 298 K, δ): 197.8, 147.8, 138.1, 131.0, 130.8, 130.2, 125.9, 124.8, 124.7, 116.4, 104.4, 47.8, 26.5, 25.6.

### Experimental Example 1

Confirmation of Absorbing Properties of Two-Photon Absorbing Fluorophores

The inventors examined the absorbing properties of two-photon absorbing fluorophores of the present invention, and the results are shown in FIGS. 2 to 8, 31, 33, and 40.

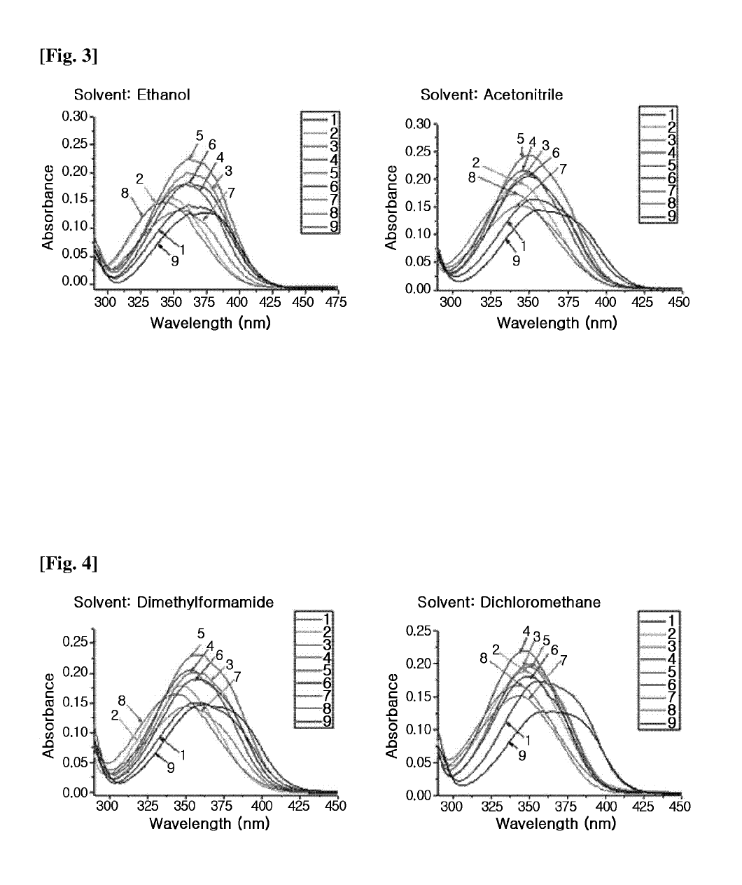

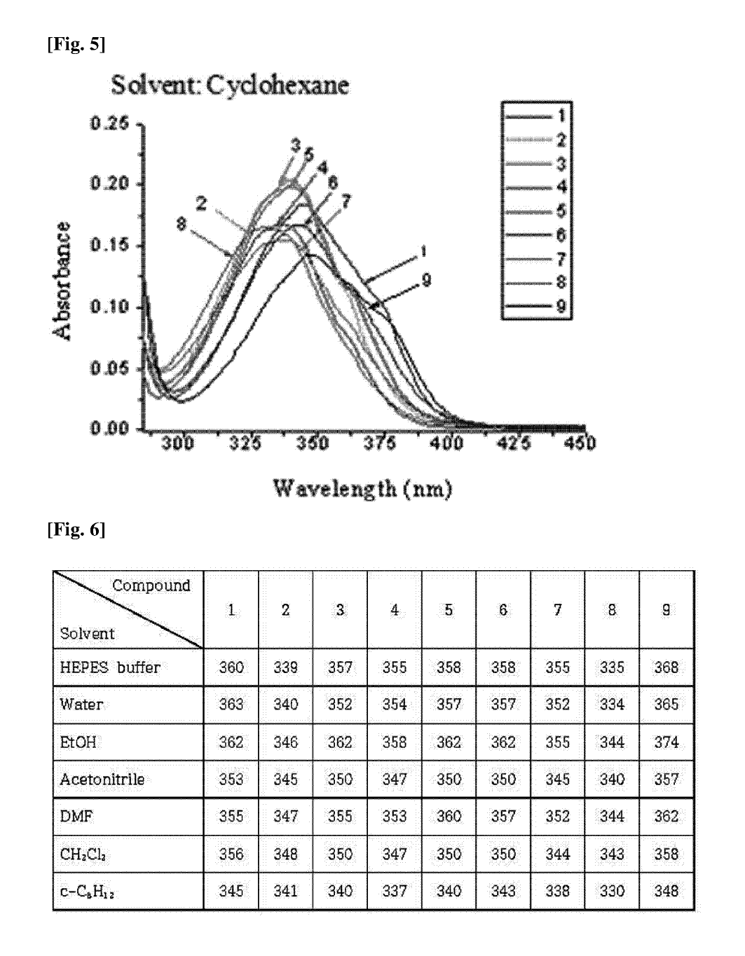

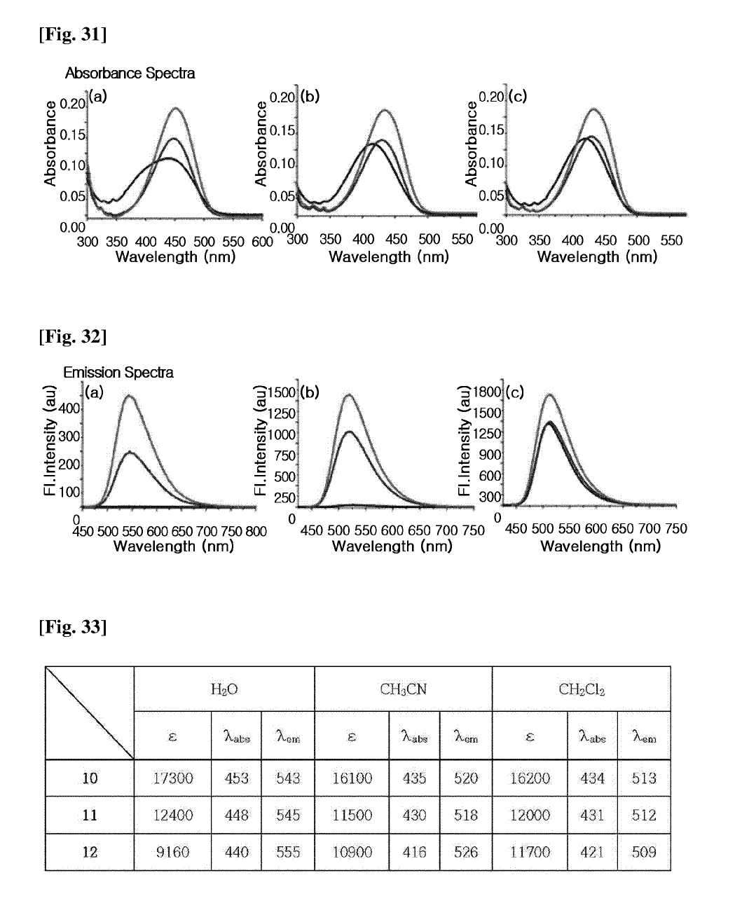

Specifically, to confirm the absorbing properties of two-photon absorbing fluorophores, the inventors measured absorbance spectra for Compounds 1 to 9 at the concentration of 10 μM in HEPES buffer (containing 1% DMSO, pH 7.4) and water (containing 1% DMSO), contained in quartz cells with a light path length of 1 cm, and the results are respectively shown in the left and right graphs of FIG. 2. Absorbance spectra for Compounds 1 to 9 at the concentration of 10 μM in ethanol and acetonitrile were measured, and the results are respectively shown in the left and right graphs of FIG. 3. Absorbance spectra for Compounds 1 to 9 at the concentration of 10 μM in dimethylformamide and dichloromethane were measured, and the results are respectively shown in the left and right graphs of FIG. 4. Absorbance spectra for Compounds 1 to 9 at the concentration of 10 μM in cyclohexane were measured, and the result is shown in FIG. 5. Absorbance spectra for Compounds 10 to 12 at the concentration of 10 μM in water (a, containing 1% DMSO), acetonitrile (b), and dichloromethane (c) were measured, and the results are shown in FIG. 31. The absorbance spectra were measured using a HP 8453 UV/Vis spectrophotometer.

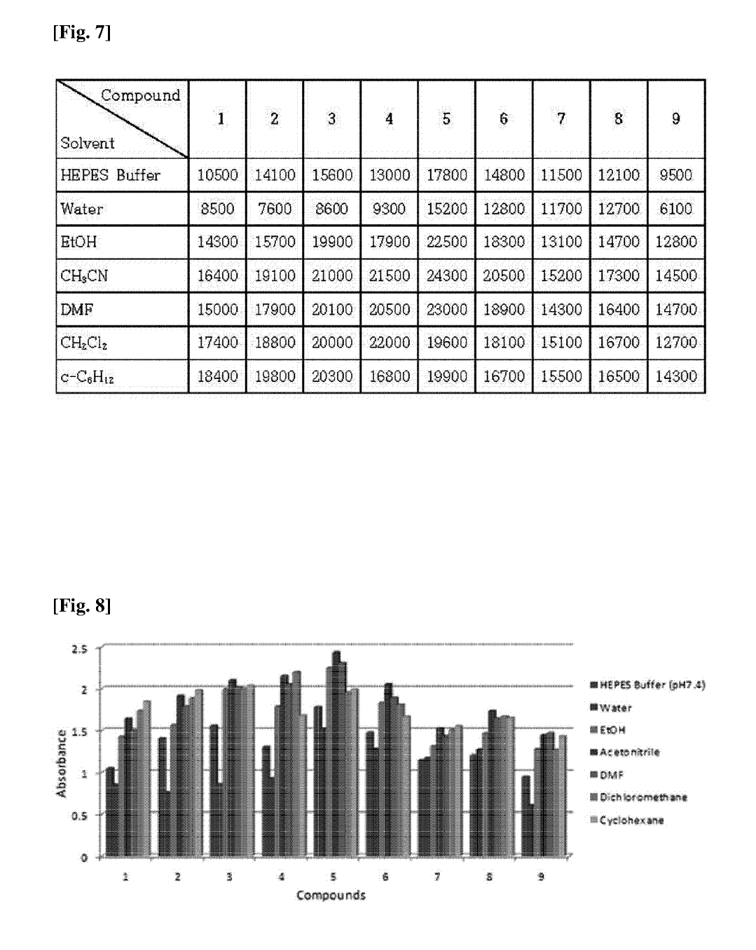

Further, molar extinction coefficients of Compounds 1 to 9 in HEPES buffer (containing 1% DMSO, pH 7.4), water (containing 1% DMSO), ethanol, acetonitrile, dimethylformamide, dichloromethane, and cyclohexane at the maximum absorption wavelength (FIG. 6) were calculated, and the results are shown in FIG. 7. Molar extinction coefficients of Compounds 10 to 12 in water (containing 1% DMSO), acetonitrile, and dichloromethane at the maximum absorption wavelength (FIG. 6) were calculated, and the results are shown in FIG. 33. Absorption spectra for Compounds 1 to 9 at the concentration of 10 μM in HEPES buffer (containing 1% DMSO, pH 7.4), water (containing 1% DMSO), ethanol, acetonitrile, a dimethylformamide, dichloromethane, and cyclohexane are shown in FIG. 8. Referring to FIG. 7, it can be confirmed that molar extinction coefficients of the two-photon absorbing fluorophores (particularly, Compounds 3 to 8) of the present invention in HEPES buffer (containing 1% DMSO, pH 7.4) and water (containing 1% DMSO) are higher than that of the conventional acedan (Compound 1). In addition, referring to FIG. 33, it was confirmed that molar extinction coefficients of Compound 10 in water (containing 1% DMSO), acetonitrile, and dichloromethane are higher than those of the conventional Compound 11 (Formula 11) and Compound 12 (Formula 12). Here, Compound 11 and Compound 12 (Ghorbanian, S. et al. J. Chem. Technol. Biotechnol. 2000, 75, 1127) are compounds conventionally known as two-photon absorbing fluorophores.

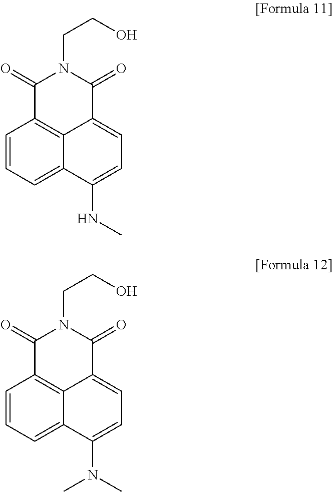 [SMILES: CNc1ccc2c3c(cccc13)C(=O)N(CCO)C2=O ; CN(C)c1ccc2c3c(cccc13)C(=O)N(CCO)C2=O]

### Experimental Example 2

Confirmation of Fluorescence Properties of Two-Photon Absorbing Fluorophores

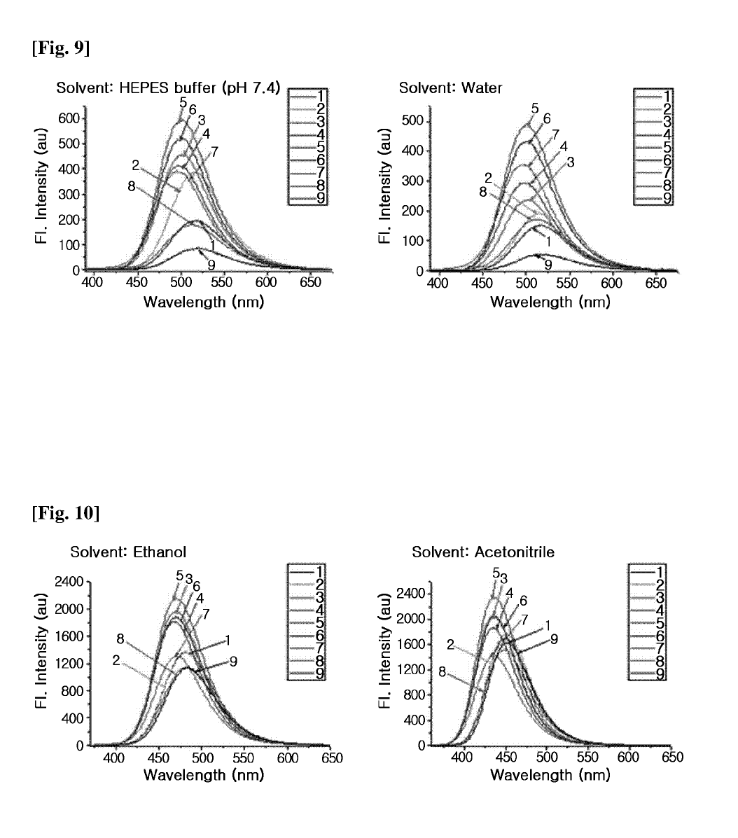

The inventors examined fluorescence properties of two-photon absorbing fluorophores of the present invention, and the results are shown in FIGS. 9 to 15, 32, 34, and 40.

Specifically, to confirm the fluorescence properties of two-photon absorbing fluorophores, the inventors measured fluorescence spectra for Compounds 1 to 9 at the concentration of 10 μM in HEPES buffer (containing 1% DMSO, pH 7.4) and water (containing 1% DMSO), contained in quartz cells with a light path length of 1 cm, and the results are respectively shown in the left and right graphs of FIG. 9.

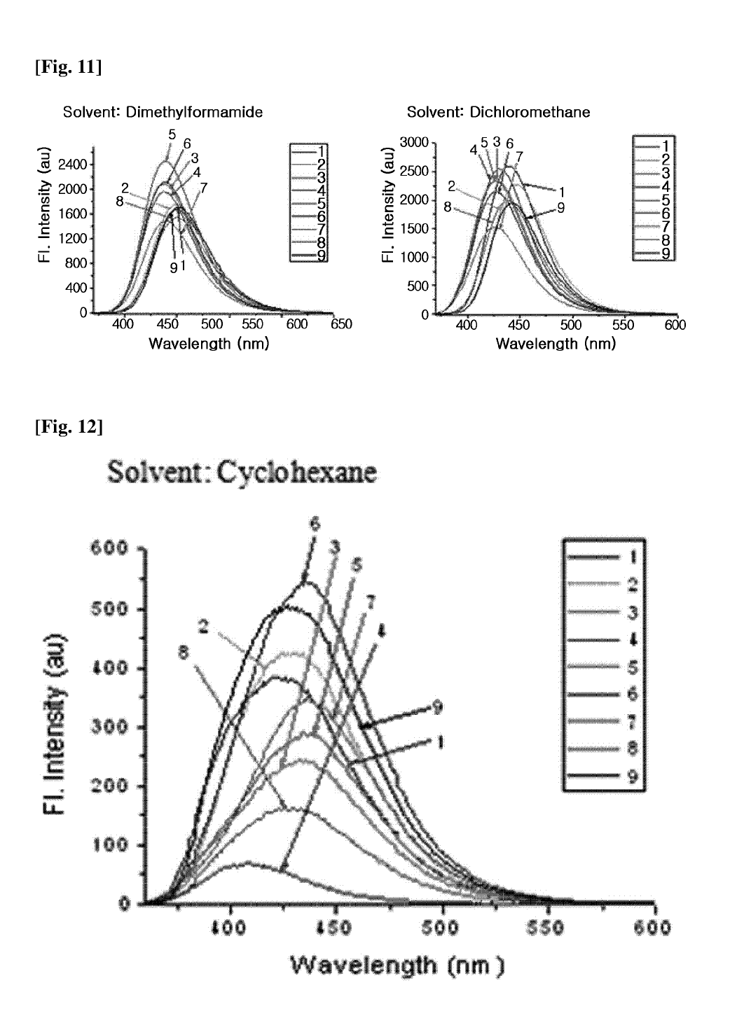

Fluorescence spectra for Compounds 1 to 9 at the concentration of 10 μM in ethanol and acetonitrile, and the results are respectively shown in the left and right graphs of FIG. 10. Fluorescence spectra for Compounds 1 to 9 at the concentration of 10 μM in dimethylformamide and dichloromethane were measured, and the results are respectively shown in the left and right graphs of FIG. 11. Fluorescence spectra for Compounds 1 to 9 at the concentration of 10 μM in cyclohexane were measured, and the results are shown in FIG. 12. Fluorescence spectra for Compounds 10 to 12 at the concentration of 10 μM in water (a, containing 1% DMSO), acetonitrile (b), and dichloromethane (c) were measured, and the results are shown in FIG. 32. All fluorescence spectra were measured at the maximum emission wavelength (FIG. 14). The fluorescence spectra were measured using a Photon Technical International Fluorescence System.

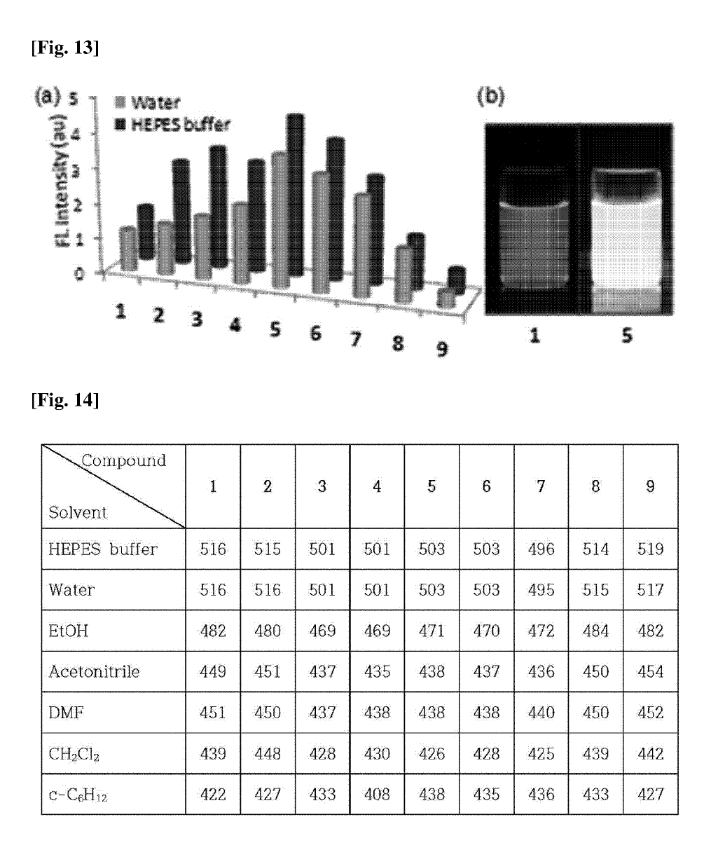

Further, fluorescence intensities for Compounds 1 to 9 at the concentration of 10 μM in HEPES buffer (containing 1% DMSO, pH 7.4) and water (containing 1% DMSO) were compared, and the results are shown in FIG. 13(a). Fluorescence images (under ultra-violet box, 365 nm) for Compounds 1 and 5 at the concentration of 10 μM in water (a, containing 1% DMSO) are shown in FIG. 13 (b, left) and FIG. 13 (b, right). Referring to FIGS. 9 and 13, it can be confirmed that fluorescence intensities of the two-photon absorbing fluorophores (particularly, Compounds 2 to 8) of the present invention in HEPES buffer (containing 1% DMSO, pH 7.4) and water (containing 1% DMSO) are higher than those of the conventional acedan (Compound 1).

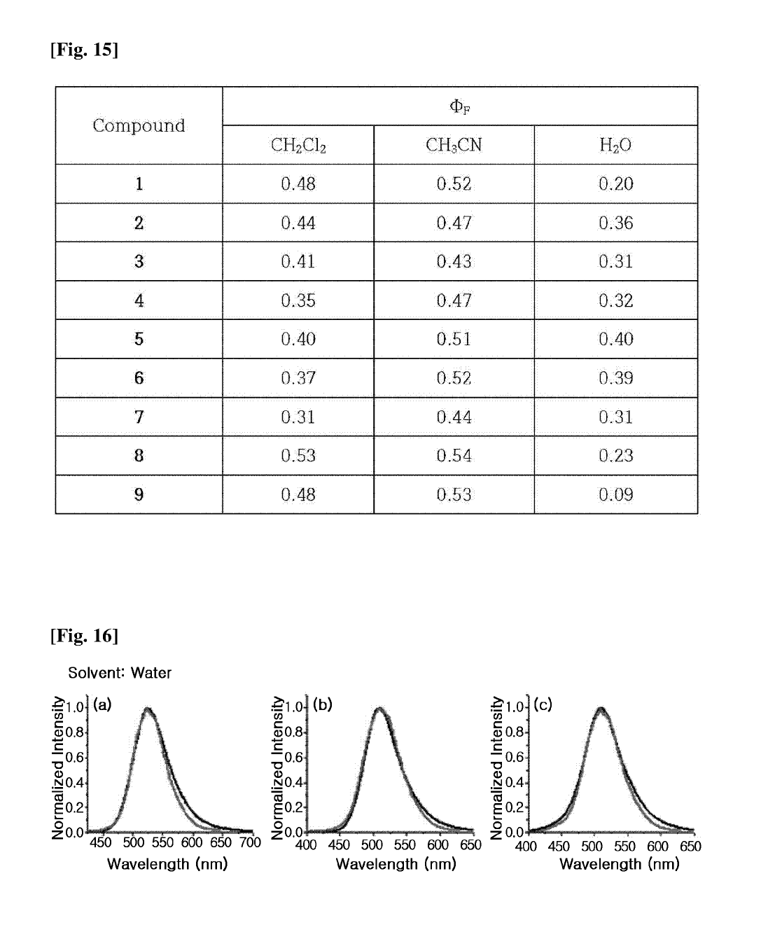

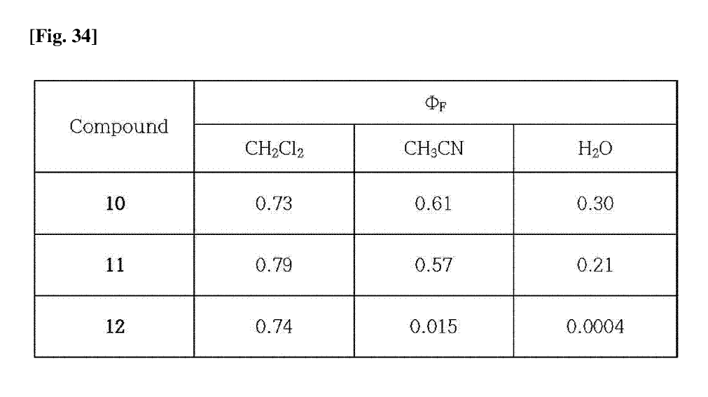

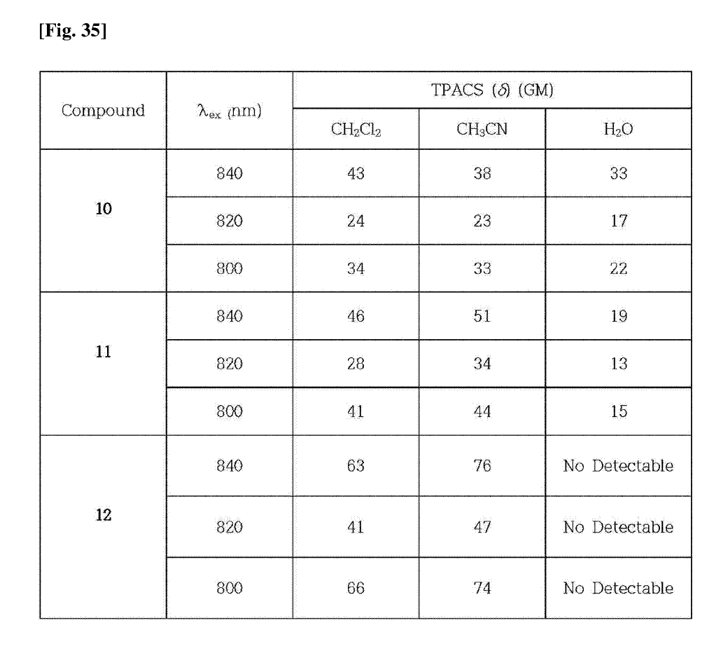

Further, fluorescence quantum yields of Compounds 1 to 9 in dichloromethane, acetonitrile, and water (containing 1% DMSO) were measured, and the results are shown in FIG. 15. Fluorescence quantum yields of Compounds 10 to 12 in dichloromethane, acetonitrile, and water (containing 1% DMSO) were measured, and the results are shown in FIG. 34. As a reference compound, rhodamine B was used (fluorescence quantum yield, Φ_{F}=0.6, measured in ethanol). Referring to FIG. 15, it can be confirmed that fluorescence quantum yields of two-photon absorbing fluorophores (particularly, Compounds 2 to 8) of the present invention in water (containing 1% DMSO) are higher than that of the conventional acedan (Compound 1). Referring to FIG. 34, it can be confirmed that a fluorescence quantum yield of Compound 10 of the present invention in water (containing 1% DMSO) is higher than those of the conventional Compounds 11 and 12.

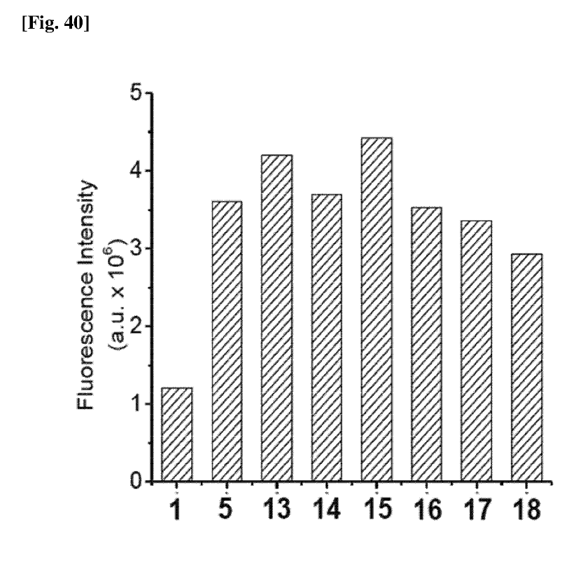

In addition, to compare the fluorescence intensities of the compounds in aqueous solution according to a structural change, fluorescence intensities of Compounds 1, 5, and 13 to 18 at the concentration of 1 μM in water (containing 1% DMSO) were compared, and the results are shown in FIG. 40. The fluorescence intensities were measured through excitation of each compound at the maximum absorption wavelength. It can be confirmed that fluorescence intensities are higher than that of the conventional acedan (Compound 1) as the rotational degrees of freedom of Compounds 13 to 18 are reduced. It can be confirmed that, as the degrees of hydrogen bonding of water molecule to the nitrogen atom in Compounds 13 to 17 are reduced, the fluorescence intensities increase. Compared to the acedan (Compound 1), it can be confirmed that the increase of fluorescence intensity of Compound 18 is caused by the decrease in allylic strain due to a pentagonal pyrrolidine ring.

### Experimental Example 3

Confirmation of the Properties of Fluorescence Due to Two-Photon Excitation of the Two-Photon Absorbing Fluorophores

The inventors examined the properties of fluorescence due to two-photon excitation of the two-photon absorbing fluorophores of the present invention, and the results are shown in FIGS. 16 to 29 and 35.

Specifically, to confirm the fluorescence properties of the two-photon absorbing fluorophores under two-photon excitation, the inventors measured fluorescence spectra by two-photon excitation using a titanium:sapphire oscillator (Ti:sapphire oscillator), which was pumped by a frequency-doubled neodimium:yttrium orthovanadate laser (Nd:YVO4 laser; Verdi, Coherent) with an output power of 5.0 W. Output pulse energy was 40 nJ, and repetition rate was 380 kHz.

After quartz cells with a light path length of 1 mm were charged with Compound 1 at the concentration of 10 μM in water (containing 1% DMSO), fluorescence spectra by one-photon (black) and two-photon (red) excitation were measured at 740 nm (a), 760 nm (b), and 780 nm (c), and the results are shown in FIG. 16. For Compound 1 at the concentration of 10 μM in acetonitrile, fluorescence spectra by one-photon (black) and two-photon (red) excitation were measured at 740 nm (a), 760 nm (b), and 780 nm (c), and the results are shown in FIG. 17. For Compound 1 at the concentration of 10 μM in dichloromethane, fluorescence spectra by one-photon (black) and two-photon (red) excitation were measured at 740 nm (a), 760 nm (b), and 780 nm (c), and the results are shown in FIG. 18.

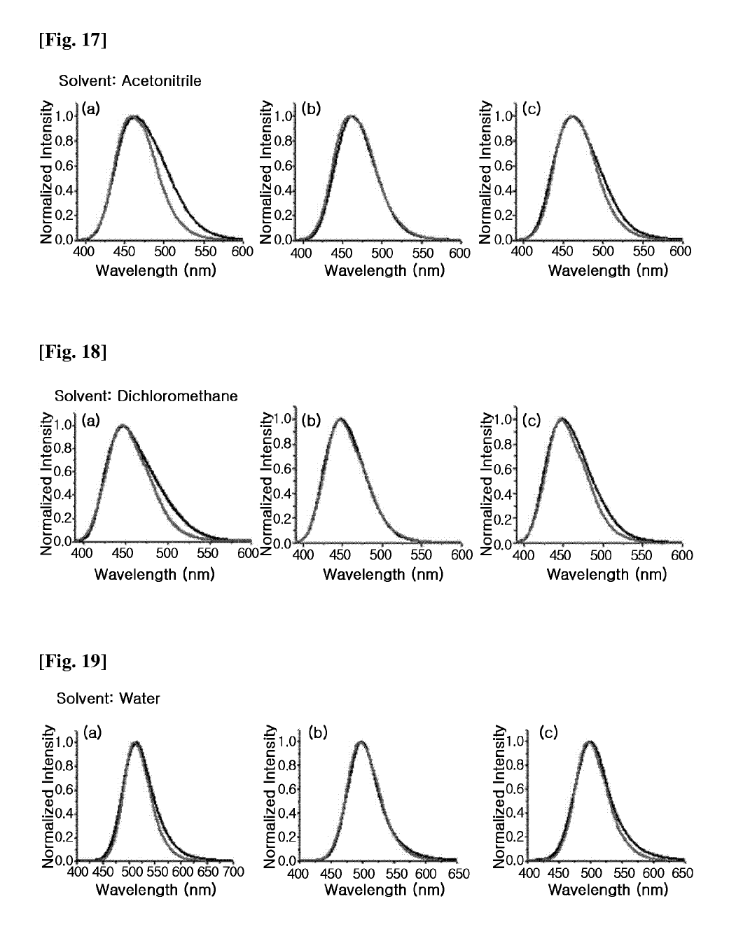

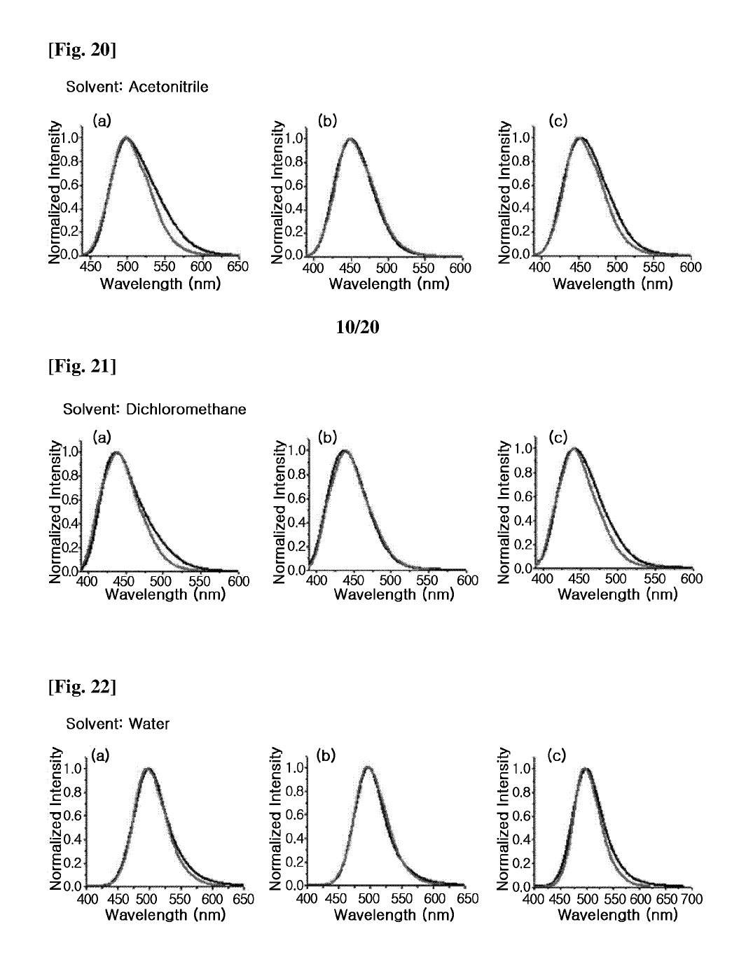

For Compound 5 at the concentration of 10 μM in water (containing 1% DMSO), fluorescence spectra by one-photon (black) and two-photon (red) excitation were measured at 740 nm (a), 760 nm (b), and 780 nm (c), and the results are shown in FIG. 19. For Compound 5 at the concentration of 10 μM in acetonitrile, fluorescence spectra by one-photon (black) and two-photon (red) excitation were measured at 740 nm (a), 760 nm (b), and 780 nm (c), and the results are shown in FIG. 20. For Compound 5 at the concentration of 10 μM in dichloromethane, fluorescence spectra by one-photon (black) and two-photon (red) excitation were measured at 740 nm (a), 760 nm (b), and 780 nm (c), and the results are shown in FIG. 21.

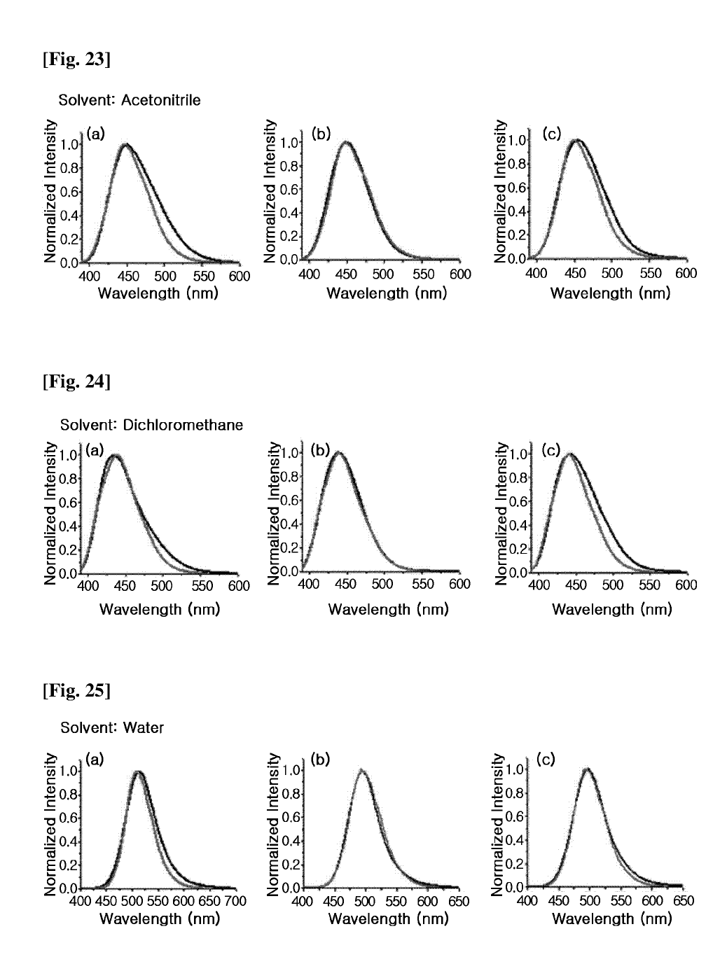

For Compound 6 at the concentration of 10 μM in water (containing 1% DMSO), fluorescence spectra by one-photon (black) and two-photon (red) excitation were measured at 740 nm (a), 760 nm (b), and 780 nm (c), and the results are shown in FIG. 22. For Compound 6 at the concentration of 10 μM in acetonitrile, fluorescence spectra generated by one-photon (black) and two-photon (red) excitation were measured at 740 nm (a), 760 nm (b), and 780 nm (c), and the results are shown in FIG. 23. For Compound 6 at the concentration of 10 μM in dichloromethane, fluorescence spectra by one-photon (black) and two-photon (red) excitation were measured at 740 nm (a), 760 nm (b), and 780 nm (c), and the results are shown in FIG. 24.

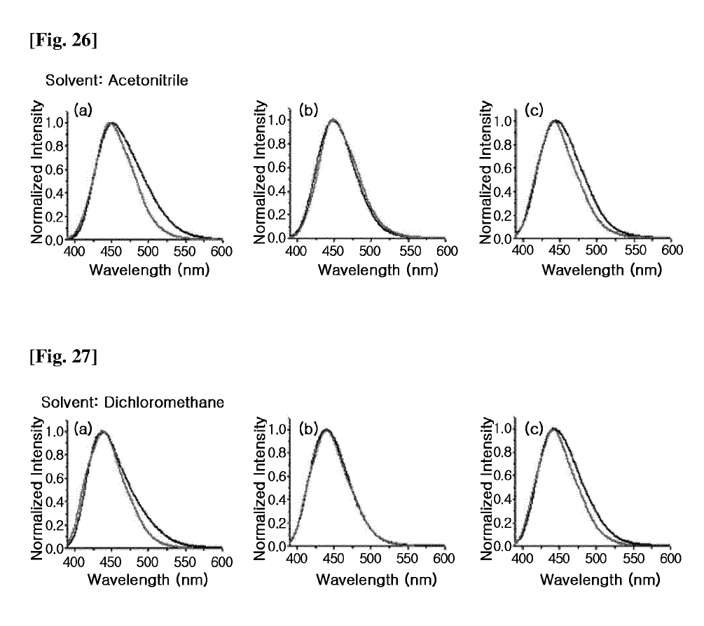

For Compound 7 at the concentration of 10 μM in water (containing 1% DMSO), fluorescence spectra by one-photon (black) and two-photon (red) excitation were measured at 740 nm (a), 760 nm (b) and 780 nm (c), and the results are shown in FIG. 25. For Compound 7 at the concentration of 10 μM in acetonitrile, fluorescence spectra by one-photon (black) and two-photon (red) excitation were measured at 740 nm (a), 760 nm (b), and 780 nm (c), and the results are shown in FIG. 26. For Compound 7 at the concentration of 10 μM in dichloromethane, fluorescence spectra by one-photon (black) and two-photon (red) excitation were measured at 740 nm (a), 760 nm (b), and 780 nm (c), and the results are shown in FIG. 27.

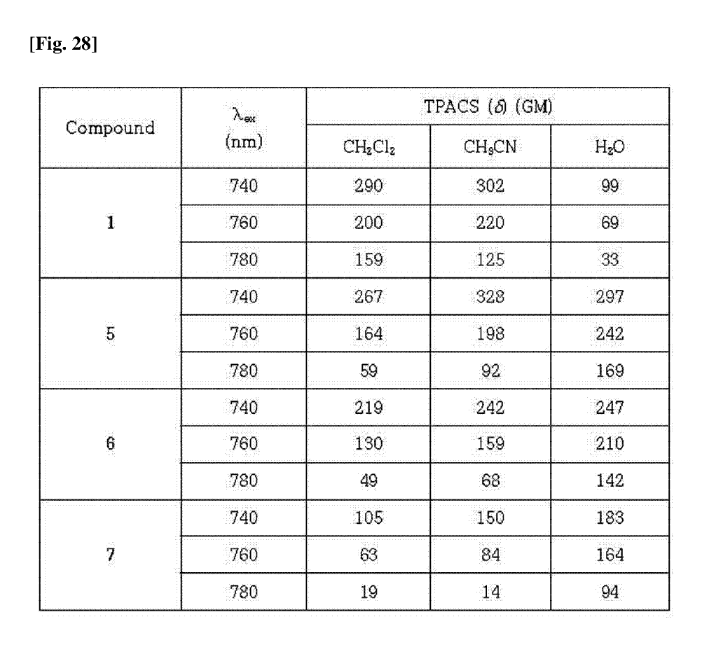

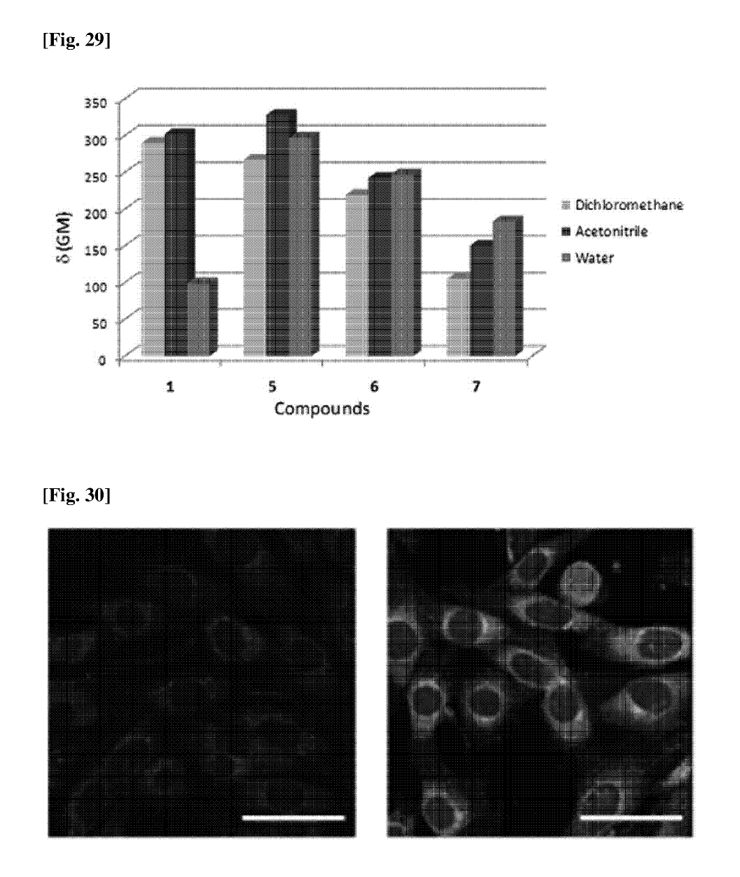

In addition, two-photon absorption cross-section values of each of Compounds 1 and 5 to 7 in dichloromethane solution, acetonitrile solution, and water (containing 1% DMSO) were measured, and the results are shown in FIGS. 28 and 29. Two-photon absorption cross-section values of each of Compounds 10 to 12 in a dichloromethane solution, an acetonitrile solution, and water (containing 1% DMSO) were measured, and the results are shown in FIG. 35. As a comparative compound, rhodamine B generally used as a fluorescent probe was used (two-photon absorption cross-section values, GM=300 (740 nm), 470 (760 nm) and 540 (780 nm), measured in an ethanol solution). Two-photon absorption cross-section values were measured by a two-photon induced fluorescence method (Fischer, A. et al. Applied Optics 1995, 34, 1989), 1 GM refers to 10⁻⁵⁰ cm⁴ s photon⁻¹ molecule⁻¹. Referring to FIGS. 28 and 29, it can be confirmed that two-photon absorption cross-section values of the two-photon absorbing fluorophores (Compounds 5 to 7) of the present invention in water (containing 1% DMSO) were higher than that of the conventional acedan (Compound 1). Referring to FIG. 35, it can be confirmed that the two-photon absorption cross-section value of Compound 10 of the present invention in water (containing 1% DMSO) is higher than those of the conventional Compounds 11 and 12.

### Experimental Example 4

Observation of Two-Photon Fluorescence Microscopic Images of NIH3T3 Cells Treated with Compounds 1 and 5

The inventors observed fluorescence changes after NIH3T3 cells were treated with the conventional acedan (Compound 1) and Compound 5 through two-photon microscopy, and the results are shown in FIG. 30.

Specifically, NIH3T3 cells were prepared in a 60 mm dish at a density of 2×10⁶ cells/dish. The cells were cultured in Dulbecco's Modified Eagles Medium (DMEM, Hyclone) with 10% fetal bovine serum (Hyclone) and 1% antibiotics (WelGENE) in a 5% CO₂-95% air atmosphere at 37° C. For cellular imaging, each cell dish was treated with Compounds 1 and 5 at the concentration of 50 μM, stored under the same conditions described above for 30 minutes, and observed using a two-photon microscope. Before fluorescence measurement, any amount of a compound that did not penetrate into the cells was removed by pipette suction, and a phosphate buffer saline (PBS) buffer solution was added. The two-photon microscope was composed of an upright microscope (BX51, Olympus) and a 20× objective lens (HCX APO, 11507751, NA 1.0, Leica) and used a titanium (Ti):sapphire laser (Chameleon Ultra II, Coherent) with a power of 50 mW at a two-photon excitation wavelength of 760 nm. Referring to FIG. 29, it can be confirmed that the two-photon fluorescence microscopic image of the two-photon absorbing fluorophore (Compound 5) of the present invention provides an image that is clearer than that of the conventional acedan (Compound 1).

### Experimental Example 5

Observation of Two-Photon Fluorescence Microscopic Images of HeLa Cells Treated with Compounds 1, 5, 10, and 12

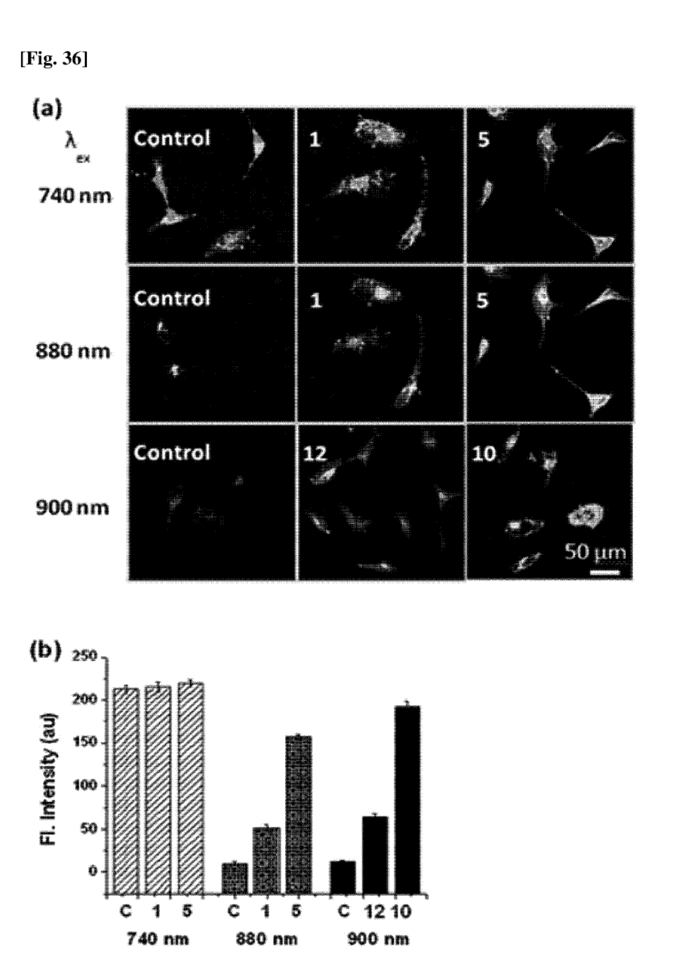

The inventors observed fluorescence changes after HeLa cells were treated with Compounds 1, 5, 10, and 12 of the present invention through two-photon microscopy, and the results are shown in FIG. 36.

Specifically, the HeLa cells were prepared in a 60 mm dish at a density of 2×10⁴ cells/dish. The cells were cultured in a DMEM (Hyclone) with 10% fetal bovine serum (Hyclone) and penicillin-streptomycin (Hyclone) in a 5% CO₂-95% air atmosphere at 37° C. For cellular fluorescence imaging, each cell dish was treated with Compounds 1, 5, 10, and 12 at the concentration of 100 μM, stored under the same storage conditions as described above for 30 minutes, and observed using a two-photon microscope. Before a fluorescence measurement, any amount of compound that did not penetrate into the cells was removed by pipette suction, and the cells were washed with a PBS buffer solution three times and fixed with 4% paraformaldehyde for 10 minutes. The two-photon microscope was composed of an upright microscope (BX51, Olympus) and 20× and 40× objective lenses (XLUMPLEN, NA 1.0, Olympus) and used a titanium:sapphire laser (Ti:Sapphire laser; Chameleon Ultra II, Coherent) outputting a laser power of 160 mW at two-photon excitation wavelengths of 740 nm (Compounds 1 and 5), 880 nm (Compounds 1 and 5) and 900 nm (Compounds 10 and 12). Referring to FIG. 36, it can be confirmed that the two-photon fluorescence microscopic image of the two-photon absorbing fluorophore (Compound 5) of the present invention is clearer than that of the conventional acedan (Compound 1). In addition, it can be confirmed that the two-photon fluorescence microscopic image of Compound 10 of the present invention provides an image that is clearer than that of the conventional Compound 12.

### Experimental Example 6

Observation of Two-Photon Fluorescence Microscopic Images of Mouse Tissues Treated with Compounds 1, 5, 10, and 12

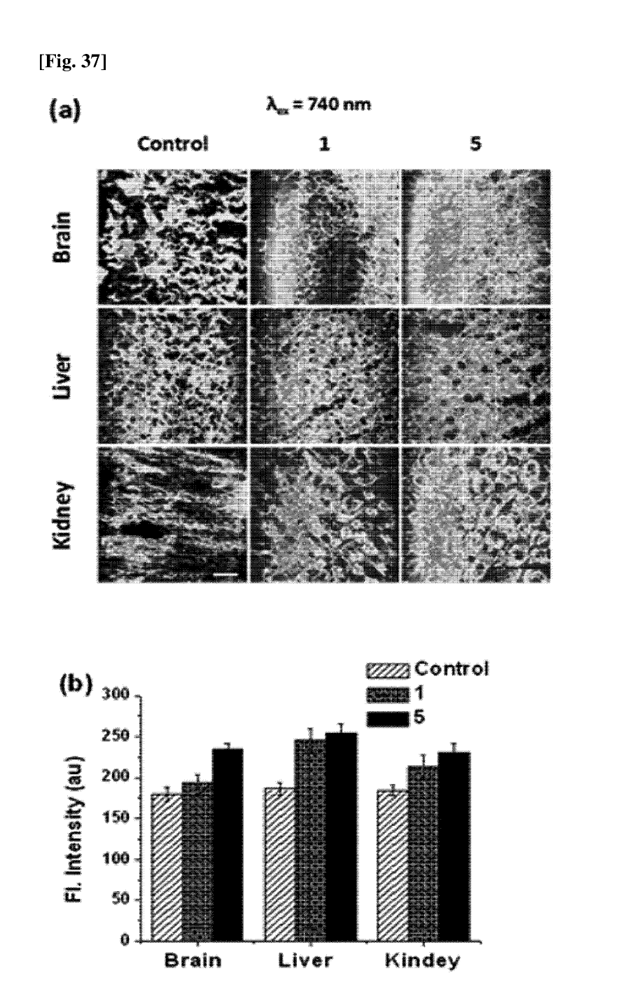

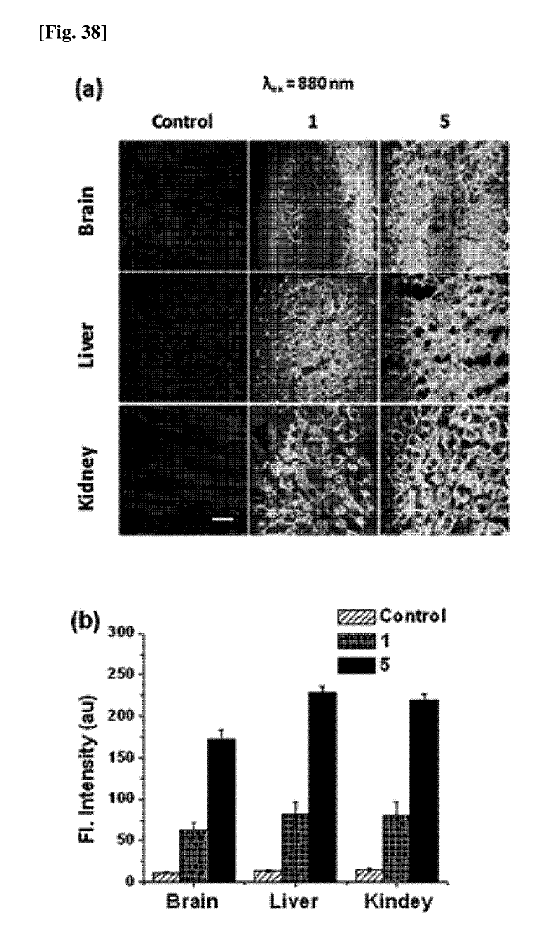

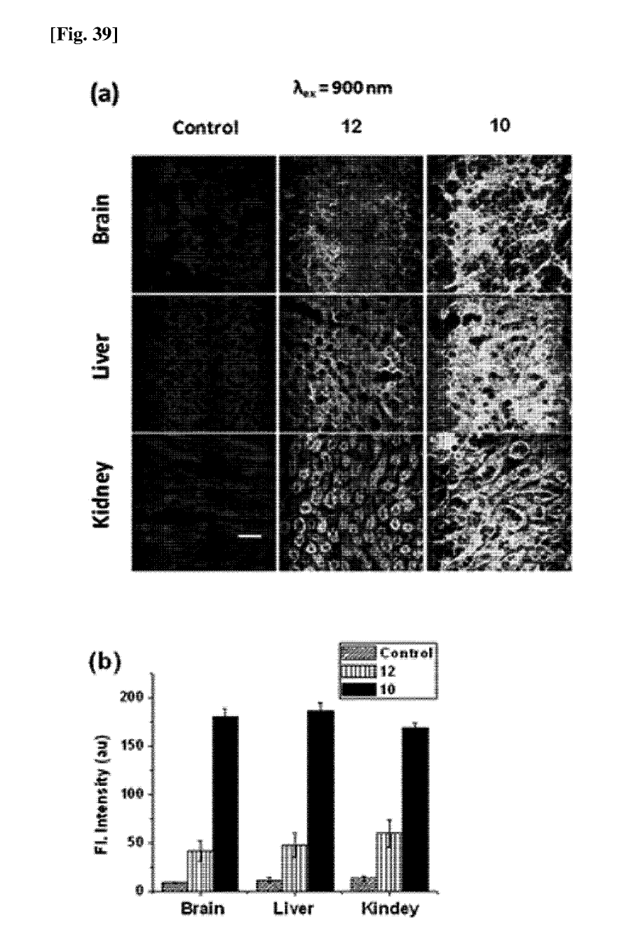

The inventors observed fluorescence changes in mouse tissues treated with Compounds 1, 5, 10, and 12 of the present invention using a two-photon microscope, and the results are shown in FIGS. 37, 38, and 39.

Specifically, a C57BL6 mouse (5-week-old, male, SAMTAKO Co.) was used, and an experiment was performed under a light-protected condition (dark room). The brain, liver and kidney of the mouse were extracted, washed with a PBS buffer solution, and frozen with dry-ice for 5 minutes. The frozen organs were shattered with a hammer and cut to a thickness of 16 μm using a slicer (cryostat machine, Leica, CM3000 model), thereby preparing a tissue slice sample. To fix the organs onto the slicer, an optical cutting temperature (OCT) compound, 10% polyvinyl alcohol, 25% polyethylene glycol, and 85.5% inactive species were used. The tissue slice sample was mounted on a specimen block (Paul Marienfeld GMbH & Co.), the specimen block was immersed in 4% paraformaldehyde for 10 minutes and washed with a PBS buffer solution, and the tissue was fixed again using a mounting solution (Gel Mount, BIOMEDA). The prepared tissue slice sample was immersed in PBS buffer of the concentration of 100 μM Compounds 1, 5, 10, and 12 for 10 minutes, washed with PBS buffer three times, and fixed in 4% paraformaldehyde. The two-photon microscope was composed of an upright microscope (BX51, Olympus) and 20× and 40× objective lenses (XLUMPLEN, NA 1.0, Olympus) and used a Ti:Sapphire laser (Chameleon Ultra II, Coherent) with a power of 120 mW at two-photon excitation wavelengths of 740 nm (Compounds 1 and 5), 880 nm (Compounds 1 and 5) and 900 nm (Compounds 10 and 12). Referring to FIGS. 37 to 39, it can be confirmed that the two-photon fluorescence microscopic image of the two-photon absorbing fluorophore (Compound 5) of the present invention provides an image that is clearer than that of the conventional acedan (Compound 1) in a suitable biological optical window range (800-1000 nm). In addition, it can be confirmed that the two-photon fluorescence microscopic image of Compound 10 of the present invention is clearer than that of the conventional Compound 12.

It would be understood by those of ordinary skill in the art that the above descriptions of the present invention are exemplary, and the exemplary embodiments disclosed herein can be easily modified into other specific forms without changing the technical spirit or essential features of the present invention. Therefore, the exemplary embodiments described above should be interpreted as illustrative and not limited in any aspect.

### INDUSTRIAL APPLICABILITY

Since one-photon or two-photon absorbing fluorophores of the present invention have much higher fluorescence quantum yield and two-photon absorption cross-section values in aqueous solution, compared to those of the conventional fluorophores, the new dyes are is expected to be highly promising to use for bioimaging research, especially under two-photon microscopy.

1. Compounds represented by Formula 1 or a pharmaceutically acceptable salts thereof: 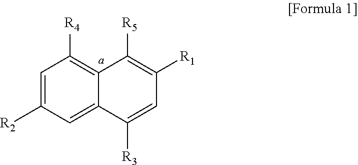
wherein R₁ is hydrogen or 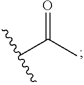 [SMILES: *C(C)=O]
R₂ is hydrogen, 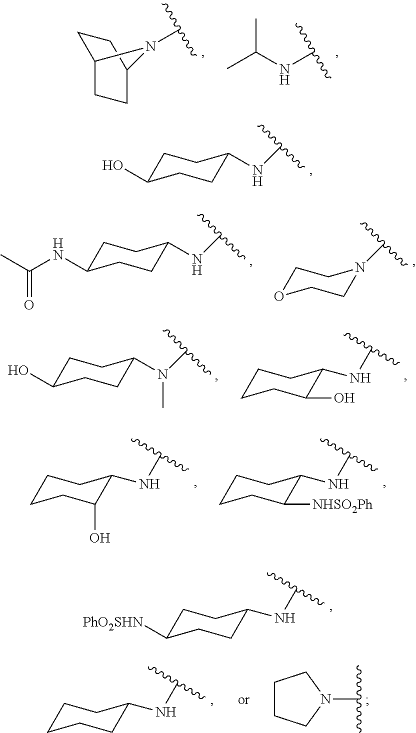
R₃ is hydrogen or 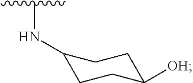 [SMILES: *NC1CCC(O)CC1]
R₄ and R₅ are independently hydrogen or R₄ and R₅ form 6-membered heterocycle together with the carbon to which they are attached and the carbon at a site, and the heterocycle is

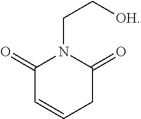 [SMILES: O=C1C=CCC(=O)N1CCO]

2. The compounds of claim 1, wherein the compounds are one-photon absorbing fluorophores or two-photon absorbing fluorophores.

3. A method for cellular imaging using the compounds of claim 1 or a pharmaceutically acceptable salts thereof.

4. A method for preparing a compound of Formula 2, comprising:
1) synthesizing 4-(6-bromonaphthalene-2-ylamino)cyclohexanol by adding trans-4-aminocyclohexanol and sodium metabisulfite to 6-bromo-2-naphthol;
2) synthesizing 4-(6-bromonaphthalene-2-ylamino)cyclohexyl methanesulfonate by adding triethylamine and methanesulfonylchloride to the 4-(6-bromonaphthalene-2-ylamino)cyclohexanol;
3) synthesizing 7-(6-bromonaphthalene-2-yl)7-azacyclo[2.2.1]heptane by adding dimethylformamide to the 4-(6-bromonaphthalene-2-ylamino)cyclohexyl methanesulfonate; and
4) adding palladium(II)acetate, diphenylphosphinopropane, ethyleneglycolvinylether, and triethylamine to the 7-(6-bromonaphthalene-2-yl)7-azacyclo[2.2.1]heptane.

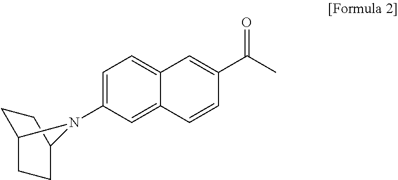 [SMILES: CC(=O)c1ccc2cc(N3C4CCC3CC4)ccc2c1]

5. A method for preparing a compound of Formula 3, comprising:
1) synthesizing 6-bromo-N-isopropylnaphthalene-2-amine by adding isopropylamine and sodium metabisulfite to 6-bromo-2-naphthol; and
2) adding palladium(II)acetate, diphenylphosphinopropane, ethyleneglycolvinylether, and triethylamine to the 6-bromo-N-isopropylnaphthalene-2-amine

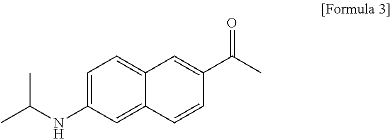 [SMILES: CC(=O)c1ccc2cc(NC(C)C)ccc2c1]

6. A method for preparing a compound of Formula 5, comprising:
1) synthesizing 4-(6-bromonaphthalene-2-ylamino)cyclohexanol by adding trans-4-aminocyclohexanol and sodium metabisulfite to 6-bromo-2-naphthol; and
2) adding palladium(II)acetate, diphenylphosphinopropane, ethyleneglycolvinylether, and triethylamine to the 4-(6-bromonaphthalene-2-ylamino)cyclohexanol.

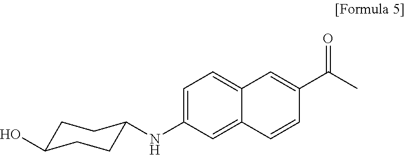 [SMILES: CC(=O)c1ccc2cc(NC3CCC(O)CC3)ccc2c1]

7. A method for preparing a compound of Formula 9, comprising:
adding a formaldehyde aqueous solution, sodium cyanoborohydride, and zinc chloride to the compound of Formula 5 of claim 6.

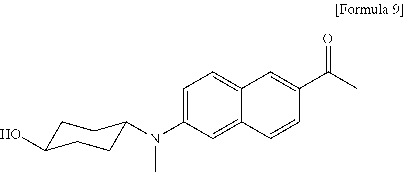 [SMILES: CC(=O)c1ccc2cc(N(C)C3CCC(O)CC3)ccc2c1]

8. A method for preparing a compound of Formula 6, comprising:
1) synthesizing 1-(6-hydroxynaphthalen-2-yl)ethanone by adding palladium(II)acetate, diphenylphosphinopropane, ethyleneglycolvinylether, and triethylamine to 6-bromo-2-naphthol;
2) synthesizing 1-(6-(4-aminocyclohexylamino)naphthalene-2-yl)ethanone by adding trans-1,4-diaminocyclohexane and sodium metabisulfite to the 1-(6-hydroxynaphthalen-2-yl)ethanone; and
3) adding acetic anhydride to the 1-(6-(4-aminocyclohexylamino)naphthalene-2-yl)ethanone.

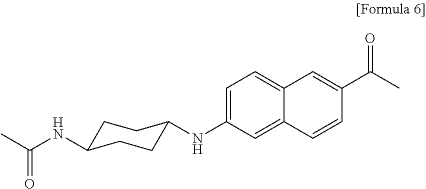 [SMILES: CC(=O)NC1CCC(Nc2ccc3cc(C(C)=O)ccc3c2)CC1]

9. A method for preparing a compound of Formula 8, comprising:
1) synthesizing 4-(6-bromonaphthalene-2-yl)morpholine by adding morpholine and sodium metabisulfite to 6-bromo-2-naphthol; and
2) adding palladium(II)acetate, diphenylphosphinopropane, ethyleneglycolvinylether, and triethylamine to the 4-(6-bromonaphthalene-2-yl)morpholine.

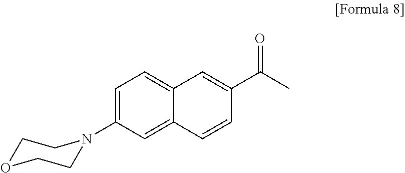 [SMILES: CC(=O)c1ccc2cc(N3CCOCC3)ccc2c1]

10. A method for preparing a compound of Formula 10, comprising:
adding trans-4-aminocyclohexanol to 6-bromo-2-(2-hydroxyethyl)-1H-benzo[de]isoquinolin-1,3(2H)-dione.

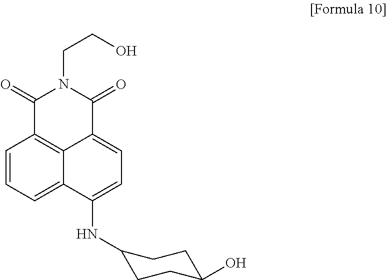 [SMILES: O=C1c2cccc3c(NC4CCC(O)CC4)ccc(c23)C(=O)N1CCO]

11. A method for preparing a compound of Formula 13, comprising:
adding sodium metabisulfite and (1S,2S)-2-aminocyclohexanol to 1-(6-hydroxynaphthalen-2-yl)ethanone.

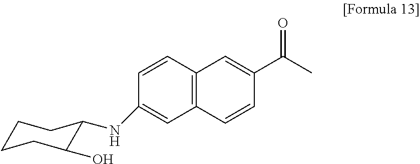 [SMILES: CC(=O)c1ccc2cc(NC3CCCCC3O)ccc2c1]

12. A method for preparing a compound of Formula 14, comprising:
adding sodium metabisulfite and (1R,2S)-2-aminocyclohexanol to 1-(6-hydroxynaphthalen-2-yl)ethanone.

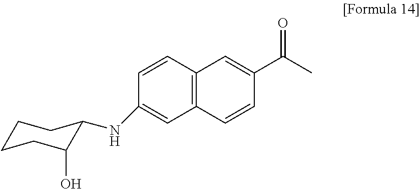 [SMILES: CC(=O)c1ccc2cc(NC3CCCCC3O)ccc2c1]

13. A method for preparing a compound of Formula 15, comprising:
1) synthesizing 1-(6-(((1S,2S)-2-aminocyclohexyl)amino)naphthalene-2-yl)ethanone by adding sodium metabisulfite and (1R,2R)-cyclohexane-1,2-diamine to 1-(6-hydroxynaphthalen-2-yl)ethanone; and
2) adding benzenesulfonyl chloride and triethylamine to the 1-(6-4(1S,2S)-2-aminocyclohexyl)amino)naphthalene-2-yl)ethanone.

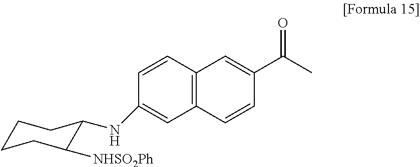

14. A method for preparing a compound of Formula 16, comprising:
1) synthesizing 1-(6-(((1R,4R)-4-aminocyclohexyl)amino)naphthalene-2-yl)ethanone by adding sodium metabisulfite and (1R,4R)-cyclohexane-1,4-diamine to 1-(6-hydroxynaphthalen-2-yl)ethanone; and
2) adding benzenesulfonyl chloride and triethylamine to the 1-(6-(((1R,4R)-4-aminocyclohexyl)amino)naphthalene-2-yl)ethanone.

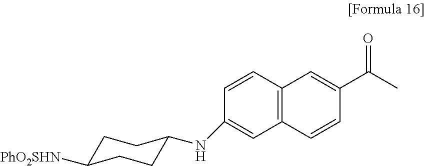

15. A method for preparing a compound of Formula 17, comprising:
adding sodium metabisulfite and cyclohexaneamine to 1-(6-hydroxynaphthalen-2-yl)ethanone.

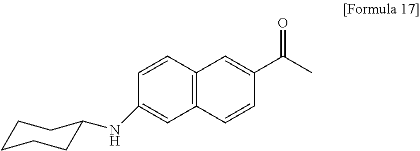 [SMILES: CC(=O)c1ccc2cc(NC3CCCCC3)ccc2c1]

16. A method for preparing a compound of Formula 18, comprising:
adding sodium metabisulfite and pyrrolidine to 1-(6-hydroxynaphthalen-2-yl)ethanone. 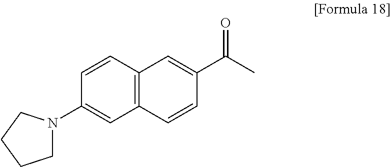 [SMILES: CC(=O)c1ccc2cc(N3CCCC3)ccc2c1]
